SEQUENCE LISTING (35 sequences)

SEQ ID NO: 1 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 2 - protein, 332 aa, Homo sapiens
SPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 3 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 4 - protein, 209 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 5 - protein, 155 aa, Homo sapiens
QLYQACMPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERCGDAVPRR

SEQ ID NO: 6 - DNA, 20 bp, Artificial Sequence
  Artificially Synthesized TNF Forward primer sequence
aaagcatgatccgagatgtg

SEQ ID NO: 7 - DNA, 20 bp, Artificial Sequence
  Artificially Synthesized TNF Reverse primer sequence
agcaggaatgagaagaggct

SEQ ID NO: 8 - DNA, 20 bp, Artificial Sequence
  Artificially Synthesized HSP70 Forward primer sequence
gtgaactacaagggcgagaa

SEQ ID NO: 9 - DNA, 22 bp, Artificial Sequence
  Artificially Synthesized HSP70 Reverse primer sequence
cgatctccttcatcttggtcag

SEQ ID NO: 10 - DNA, 21 bp, Artificial Sequence
  Artificially Synthesized SDF1 Forward primer sequence
ccgattctttgagagccatgt

SEQ ID NO: 11 - DNA, 22 bp, Artificial Sequence
  Artificially Synthesized SDF1 Reverse primer sequence
caatgcacacttgtctgttgtt

SEQ ID NO: 12 - DNA, 21 bp, Artificial Sequence
  Artificially Synthesized VEGFR2 Forward primer sequence
gacgacccattgagtccaatt

SEQ ID NO: 13 - DNA, 21 bp, Artificial Sequence
  Artificially Synthesized VEGFR2 Reverse primer sequence
gtgaggatgaccgtgtagttt

SEQ ID NO: 14 - DNA, 19 bp, Artificial Sequence
  Artificially Synthesized CX3CL1 Forward primer sequence
gcagctttccgctctgaat

SEQ ID NO: 15 - DNA, 22 bp, Artificial Sequence
  Artificially Synthesized CX3CL1 Reverse primer sequence
ctgttctgagcttccacctatc

SEQ ID NO: 16 - DNA, 23 bp, Artificial Sequence
  Artificially Synthesized CD14 Forward primer sequence
ggaaagaaactgaagcctttctc

SEQ ID NO: 17 - DNA, 19 bp, Artificial Sequence
  Artificially Synthesized CD14 Reverse primer sequence
agcaacaagccgagcataa

SEQ ID NO: 18 - DNA, 22 bp, Artificial Sequence
  Artificially Synthesized IL6 Forward primer sequence
aagagacttccagccagttgcc

SEQ ID NO: 19 - DNA, 23 bp, Artificial Sequence
  Artificially Synthesized IL6 Reverse primer sequence
actggtctgttgtgggtggtatc

SEQ ID NO: 20 - DNA, 20 bp, Artificial Sequence
  Artificially Synthesized IL8 Forward primer sequence
gcacccaaaccgaagtcata

SEQ ID NO: 21 - DNA, 21 bp, Artificial Sequence
  Artificially Synthesized IL8 Reverse primer sequence
gggacaccctttagcatcttt

SEQ ID NO: 22 - DNA, 22 bp, Artificial Sequence
  Artificially Synthesized CCL2 Forward primer sequence
gtctcagccagatgcagttaat

SEQ ID NO: 23 - DNA, 23 bp, Artificial Sequence
  Artificially Synthesized CCL2 Reverse primer sequence
ctgctggtgattctcttgtagtt

SEQ ID NO: 24 - DNA, 23 bp, Artificial Sequence
  Artificially Synthesized MMP3 Forward primer sequence
gatgatgaacgatggacagatga

SEQ ID NO: 25 - DNA, 20 bp, Artificial Sequence
  Artificially Synthesized MMP3 Reverse primer sequence
agcattggctgagtgaaaga

SEQ ID NO: 26 - DNA, 18 bp, Artificial Sequence
  Artificially Synthesized MMP9 Forward primer sequence
caacctcacggacacaca

SEQ ID NO: 27 - DNA, 21 bp, Artificial Sequence
  Artificially Synthesized MMP9 Reverse primer sequence
ctgcttctctcccatcatctg

SEQ ID NO: 28 - DNA, 22 bp, Artificial Sequence
  Artificially Synthesized Beta-actin Forward primer sequence
cttccttcctgggtatggaatc

SEQ ID NO: 29 - DNA, 21 bp, Artificial Sequence
  Artificially Synthesized Beta-actin Reverse primer sequence
ctgtgttggcatagaggtctt

SEQ ID NO: 30 - DNA, 22 bp, Artificial Sequence
  Artificially Synthesized CycA Forward primer sequence
ggctataagggttcctcctttc

SEQ ID NO: 31 - DNA, 18 bp, Artificial Sequence
  Artificially Synthesized CycA Reverse primer sequence
ttgccaccagtgccatta

SEQ ID NO: 32 - DNA, 22 bp, Artificial Sequence
  Artificially Synthesized HPRT Forward primer sequence
gacctctcgaagtgttggatac

SEQ ID NO: 33 - DNA, 21 bp, Artificial Sequence
  Artificially Synthesized HPRT Reverse primer sequence
tcaaatccctgaagtgctcat

SEQ ID NO: 34 - DNA, 20 bp, Artificial Sequence
  Artificially Synthesized YWAHZ Forward primer sequence
gacatctgcaacgacgtact

SEQ ID NO: 35 - DNA, 22 bp, Artificial Sequence
  Artificially Synthesized YWAHZ Reverse primer sequence
cggtagtagtcacccttcattt

(19) United States
(12) Patent Application Publication
(10) Pub. No.: US 2017/0218340 A1
(43) Pub. Date: Aug. 3, 2017

(54) METHOD OF CULTURING CELLS

(71) Applicant: MEDVET SCIENCE PTY LTD, Underdale, South Australia (AU)

(72) Inventors: Claudine Sharon Bonder, Adelaide (AU); Angel Francisco Lopez, Adelaide (AU)

(21) Appl. No.: 15/309,420

(22) PCT Filed: May 11, 2015
(86) PCT No.: PCT/AU2015/050230

§ 371 Date: Nov. 7, 2016

(30) Foreign Application Priority Data
May 9, 2014 (AU) 2014901720

(51) Int. Cl. C12N 5/071; A61K 35/44

(52) U.S. Cl. CPC C12N 5/0692; A61K 35/44; C12N 2501/2303; C12N 2501/125; C12N 2501/998

(57) ABSTRACT

A method for producing a population of cells, enriched for non-adherent endothelial progenitor cells (EPCs), the method comprising culturing an EPC containing population of cells in the presence of interleukin-3 (IL-3), such that a population of cells enriched for non-adherent EPCs is produced.

### RELATED APPLICATION DATA

The present application claims priority from Australian Patent Application No. 2014901720 entitled “Method of culturing cells” filed on 9 May 2014, the entire contents of which are incorporated by reference.

### SEQUENCE LISTING

The present application is filed with a Sequence Listing in electronic form. The entire contents of the Sequence Listing are hereby incorporated by reference.

### FIELD

The present disclosure relates to methods of culturing and expanding endothelial progenitor cells (EPC) and uses thereof.

### BACKGROUND

Endothelial progenitor cells (EPCs) are immature endothelial cells, which have the capacity to proliferate, migrate, and differentiate into endothelial cells but have not yet acquired characteristics of mature endothelial cells. The main source of adult EPCs is bone marrow, however EPCs can also be isolated from umbilical cord blood. EPCs are mobilized from bone marrow into peripheral blood (circulating EPCs) in response to certain physiological stimuli, such as, for example, tissue injury. Circulating EPCs were only recently identified in adult human blood and subsequent studies have suggested a role for EPCs in the maintenance of endothelial integrity and function.

Two of the more commonly studied forms of EPCs are monocytic EPCs and hemangioblastic EPCs.

Monocytic EPCs are found in peripheral blood mononuclear cells (PBMCs) and in culture are capable of forming colonies of endothelial-like cells that augment neovascularization in animal models (Hill et al N Engl J Med 2003).

Hemangioblastic EPCs circulate in peripheral blood and are also detectable in bone marrow. These cells are also mobilized from bone marrow under conditions of hypoxia, e.g., during ischemia, or in response to hematopoietic stem cell mobilization, e.g., using granulocyte colony stimulating factor (G-CSF) (Asahara et al 1997, Kawamoto and Losordo, 2008; Liu et al., 2008).

While monocytic EPCs and hemangioblastic EPCs arise from distinct lineages and show functional differences in vitro, both forms contribute to in vivo neovascularization in several disease models (Krenning et al., 2009). In this regard, EPCs have been shown to integrate into newly forming blood vessels (Asahara et al., 1997).

EPCs are not abundant in either circulating blood or the bone marrow. The low abundance of EPCs represents a critical issue to overcome in the clinical application of EPCs. (Kawamoto et al. Catheterization and Cardiovascular Interventions, 2007).

Exemplary methods for culturing and expansion of EPCs generally include the presence of fibronection, VEGF and large amounts of serum, this is undesirable as it increases the costs of EPC production. Furthermore, it is undesirable to culture in the presence of animal products (e.g. serum) since these products raise safety concerns in therapeutics.

It will be clear to the skilled person based on the foregoing that methods for culturing and expanding EPCs for clinical therapy are desirable.

### SUMMARY

In producing the present disclosure the inventors developed a method for producing a population of cells enriched for endothelial progenitor cells (EPCs). In one example, the method comprises culturing an EPC-containing population of cells in the presence of one or more of interleukin-3 (IL-3), thrombopoietin (TPO), Flt3-ligand (Flt3L), stem cell factor (SCF) and PIGF (placental growth factor). In one example, the EPCs are non-adherent EPCs.

For example, the disclosure provides a method for producing a population of cells enriched for EPCs, the method comprising culturing the EPC containing population of cells in the presence of IL-3.

In one example, the method comprises culturing the EPC containing population of cells in the presence of IL-3, such that the population of cells enriched for EPCs is produced. In this regard the inventors have shown that IL-3 provides significant growth advantage to EPCs. In one example, IL-3 provides a significant growth advantage to non-adherent EPCs.

In one example, the method additionally comprises culturing the EPC containing population of cells, in the presence of one or more factors selected from the group consisting of TPO, Flt3L and SCF.

In one example, the method additionally comprises culturing the EPC containing population of cells in the presence of placental growth factor (PIGF).

In one example, the method comprises culturing the EPC containing population of cells in a medium comprising IL-3.

In one example, the method comprises culturing the EPC containing population of cells in a medium comprising IL-3, TPO, Flt3L and SCF.

In one example, the method comprises culturing the EPC containing population of cells in a medium comprising IL-3, TPO, Flt3L, SCF and penicillin/streptomycin.

In one example, the method comprises culturing the EPC containing population of cells in a medium comprising IL-3, TPO, Flt3L, SCF and PIGF.

In one example, the method comprises culturing the EPC containing population of cells in a medium comprising IL-3, TPO, Flt3L, SCF, PIGF and penicillin/streptomycin.

In one example, the method comprises culturing the EPC containing population of cells in a medium comprising IL-3 at about 20 ng/mL, TPO at about 10 ng/mL, Flt3L at about 40 ng/mL and SCF at about 40 ng/mL, optionally PIGF at about 25 ng/mL, and optionally with an antibiotic, for example, penicillin/streptomycin.

In one example, the method further comprises adding additional IL-3 and/or factor(s) and/or medium to the EPC containing population of cells and/or a population of cells enriched for EPCs. For example, the method comprises adding additional medium following a sufficient period for EPCs in the population to expand.

In one example, the additional IL-3 and/or factor(s) and/or medium is added without removing previous IL-3 and/or factor(s) and/or medium.

In one example, the medium comprises IL-3, TPO, Flt3L and SCF.

In one example, the medium comprises IL-3, TPO, Flt3L, SCF and PIGF.

In one example, the medium is serum-free.

In one example, the EPC containing population of cells and/or the population of cells enriched for EPC is a crude population. In one example, the crude population of EPCs are non-adherent EPCs.

In one example, the EPC containing population of cells and/or the population of cells enriched for EPCs can be stored at −80° C.

In one example, the EPC containing population of cells and/or the population of cells enriched for EPC is an enriched population of EPCs. In one example, the enriched population of EPCs are non-adherent EPCs.

In one example, the EPCs are CD133+, or DSG2+, or CD34+ and CD45 dim and DSG2+.

In one example, the expanded population of cells enriched for EPCs are additionally characterised by one or more of the following CD117+, CD34+, CD133+, IL-3RA+, VEGFR2+, CD31+, CD45dim, CD144−, CD146− and CD38−. In one example, the expanded population of cells enriched for EPCs are additionally CD117+, CD34+, CD133+, IL-3RA+, VEGFR2+, CD31+, CD45dim, CD144−, CD146− and CD38−.

In one example, the expanded population of cells enriched for EPCs additionally express MHC class I and MHC class II markers.

In one example, the expanded population of cells enriched for EPCs increase expression of MHC class I in the presence of TNFα and IFNγ.

In another example, the expanded population of cells enriched for EPCs increase expression of MHC class II in the presence of TNFα.

In one example, the EPCs are smaller than at least about 6 μm, or at least about 7 μm, or at least about 8 μm, or at least about 9 μm, or at least about 10 μm, In one example, the population of EPCs are non-adherent EPCs.

In one example, the EPCs are cultured such that they remain non-adherent.

In one example, IL-3, TPO, Flt3L, SCF or PIGF are of human origin. In another example IL-3, TPO, Flt3L, SCF or PIGF are of non-human animal origin or are recombinant or fusion proteins.

In one example, the method additionally comprises testing the cells for EPC activity. In one example, the method additionally comprises testing the cells for EPC activity in vitro. Exemplary protocols for assessing EPC activity in vitro are described herein and include but are not limited to: CFU and migration assays; determining uptake of acetylated-LDL; binding of Ulex europaeus I lectin; tube formation alone or in combination with endothelial cells; secretion of angiogenic factors and cell death; proliferation and survival assays.

In one example, the method additionally comprises testing the cells for EPC function in vivo. Exemplary assessment for EPC function in vivo includes; EPC migration or EPC induction of neovascularization.

In one example, the method additionally comprises isolating the EPC containing population of cells and/or population of cells enriched for EPCs.

In one example, the EPC containing population of cells, or population of cells enriched for EPCs, are isolated by isolating CD133+ EPCs. For example, the method comprises isolating cells based on CD133 expression. Exemplary methods include contacting the cells with an agent, for example, an antibody that binds to CD133 and isolating or separating the agent and bound cells.

In one example, the EPC containing population of cells, or population of cells enriched for EPCs, are isolated by isolating DSG2+ EPCs. For example, the method comprises isolating cells based on DSG2 expression. Exemplary methods include contacting the cells with an agent, for example an antibody that binds to DSG2 and isolating or separating the agent and bound cells.

In one example, the EPC containing population of cells, or population of cells enriched for EPCs, are isolated by isolating CD34+, and CD45 dim and DSG2+ endothelial progenitor cells. For example, the method comprises isolating cells based on expression of CD34, DSG2 and low expression of CD45. Exemplary methods include contacting the cells with agents, for example antibodies that bind to CD34, DSG2 and CD45 and isolating agents that bind CD34 and DSG2 and the bound cells, and isolating cells that do not significantly bind the agent that binds CD45.

In one example, the method provides expansion of a population of cells enriched for EPCs by at least about 150 fold, or at least about 200 fold, or at least about 250 fold, or at least about 300 fold. In one example, the method provides expansion of a population of cells enriched for EPCs by at least about 100 fold. In one example, the method provides expansion of a population of cells enriched for EPCs by at least about 150 fold.

In one example, the method provides expansion of a population of cells enriched for EPCs to a therapeutically effective number. In one example, the expansion occurs within about 14 days of culture.

The present disclosure provides a method additionally comprising formulating the population of cells enriched for EPCs into a pharmaceutical formulation, for example, interleukin 3, or interleukin 8.

The present disclosure additionally provides a method of producing a pharmaceutical formulation, the method comprising obtaining a population of cells enriched for EPCs and formulating the population of cells enriched for EPCs into a pharmaceutical formulation.

The present disclosure additionally provides a method of treating a subject, the method comprising administering the population of cells enriched for EPCs or a formulation comprising a population of cells enriched for EPCs to the subject.

In one example of treating a subject, the method comprises obtaining a population of cells enriched for EPCs produced by the method or a pharmaceutical formulation comprising a population of cells enriched for EPCs and administering the population or formulation to the subject.

In one example, the subject has an EPC-associated condition.

In one example, an EPC-associated condition is characterized by insufficient EPC numbers and/or activity.

In one example, the subject has a condition characterized by insufficient neovascularization (including insufficient angiogenesis). Exemplary conditions include cardiovascular disease, cerebrovascular disease, hypertension, chronic kidney disease, vessel occlusion, diabetes, diabetic retinopathy, macular degeneration, bone healing, ischemia (including ischemia resulting from a transplant and stroke), autoimmune disease (rheumatoid arthritis, systemic lupus erythematosus, diabetes (e.g., type 1 diabetes) or systemic sclerosis), sepsis and the improvement of grafting and/or wound healing.

In one example, the subject is treated to improve grafting and/or wound healing. In the case of a graft, for example, a blood vessel graft, the population of cells enriched for EPCs can be administered immobilized on a solid support or semi-solid support, e.g., in the form of a vascular graft. Exemplary conditions to be treated by administering populations of cells enriched for EPCs include cardiovascular disease, cerebrovascular disease, hypertension, chronic kidney disease, vessel occlusion, ischemia (including stroke), an autoimmune disease, or sepsis.

In one example, the condition is coronary artery disease or dysfunctional bicuspid aortic valve.

In one example, the condition is ischemia.

In one example, the condition is stroke or a cerebro-vascular accident.

In one example, the condition is a cardiovascular disease and/or an autoimmune disease and/or an inflammatory disease.

In one example, a method for treating or preventing a condition comprises additionally administering another cell or another therapeutic compound to a subject. For example, to treat a subject suffering from diabetes (e.g., type 1 diabetes) a population enriched for EPCs according to the present disclosure are administered to a subject, e.g., in combination with pancreatic islet cells.

The present disclosure also provides a method of treating or preventing a condition associated with reduced EPC numbers or activity and/or treating or preventing a condition associated with insufficient neovascularization and/or improving grafting and/or improving wound healing in a subject, said method comprising administering to a subject in need thereof a solid support or a semi-solid support comprising the population of cells enriched for EPCs.

For example, an effective amount, e.g., a therapeutically or prophylactically effective amount of cells is administered to the subject.

The present disclosure also provides a kit comprising the medium for culturing the EPC containing population of cells to produce a population of cells enriched for EPCs described herein according to any example, optionally, packaged with instructions for use in a method as described herein. In one example, the kit contains a serum free medium and one or more of IL-3, TPO, Flt3L, SCF and PIGF. In one example, the kit comprises IL-3. In one example, the kit may also comprise agents for selection of EPCs that are either CD133+, DSG2+ or CD34+, DSG2+ and CD45dim.

The present disclosure also provides a kit for isolating and/or expanding the population of cells enriched for EPCs described herein according to any example, optionally, packaged with instructions for use in a method as described herein.

The present disclosure also provides a kit comprising a population of cells enriched for EPCs described herein according to any example, optionally, packaged with instructions for use in a method as described herein.

Described herein according to any example IL-3 comprises, for example, the sequence set forth in SEQ ID NO: 1 or a protein having, at least 40% identity thereto, or at least 50% identity thereto, or at least 60% identity thereto, or at least 70% identity thereto, or at least 80% identity thereto or at least 90% identity thereto, or at least 95% identity thereto, or at least 99% identity thereto, or at least 100% identity thereto. Described herein according to any example, IL-3 is of, for example, a concentration about 10 to 3000 ng/mL, or about 10 to 2000 ng/mL, or about 10 to 1000 ng/mL, or about 10 to 250 ng/mL, or about 10 to 25 ng/mL, or about 0.25 to 25 ng/mL, or about 20 ng/mL.

Described herein according to any example TPO comprises, for example, the sequence set forth in SEQ ID NO: 2 or a protein having, at least 40% identity thereto, or at least 50% identity thereto, or at least 60% identity thereto, or at least 70% identity thereto, or at least 80% identity thereto or at least 90% identity thereto, or at least 95% identity thereto, or at least 99% identity thereto, or at least 100% identity thereto. Described herein according to any example, TPO is of, for example, a concentration about 0.1 to 3000 ng/ml, or about 0.1 to 1000 ng/mL, or about 1 to 500 ng/mL, or about 1 to 250 ng/mL, or about 1 to 200 ng/mL, or about 1 to 100 ng/mL, or about 1 to 50 ng/mL, or about 10 ng/mL.

Described herein according to any example SCF comprises, for example, the sequence set forth in SEQ ID NO: 3 or a protein having, at least 40% identity thereto, or at least 50% identity thereto, or at least 60% identity thereto, or at least 70% identity thereto, or at least 80% identity thereto or at least 90% identity thereto, or at least 95% identity thereto, or at least 99% identity thereto, or at least 100% identity thereto. Described herein according to any example, SCF is of, for example, a concentration about 0.1 to 2000 ng/mL, or about 0.1 to 1000 ng/mL, or about 1 to 500 ng/mL, or about 10 to 200 ng/mL, or about 20 to 100 ng/mL, or about 30 to 50 ng/mL, or about 40 ng/mL.

Described herein according to any example Flt3L comprises, for example, the sequence set forth in SEQ ID NO: 4 or a protein having, at least 40% identity thereto, or at least 50% identity thereto, or at least 60% identity thereto, or at least 70% identity thereto, or at least 80% identity thereto or at least 90% identity thereto, or at least 95% identity thereto, or at least 99% identity thereto, or at least 100% identity thereto. Described herein according to any example, FLt3L is of, for example, a concentration about 0.1 to 2000 ng/mL, or about 0.1 to 1000 ng/mL, or about 1 to 500 ng/mL, or about 10 to 200 ng/mL, or about 20 to 100 ng/mL, or about 30 to 50 ng/mL, or about 40 ng/mL.

Described herein according to any example PIGF comprises, for example, the sequence set forth in SEQ ID NO: 5 or a protein having, at least 40% identity thereto, or at least 50% identity thereto, or at least 60% identity thereto, or at least 70% identity thereto, or at least 80% identity thereto or at least 90% identity thereto, or at least 95% identity thereto, or at least 99% identity thereto, or at least 100% identity thereto. Described herein according to any example, PIGF is of, for example, a concentration about 0.1 to 3000 ng/mL, or about 0.1 to 2000 ng/mL, or about 1 to 1000 ng/mL, or about 1 to 500 ng/mL, or about 1 to 250 ng/mL, or about 10 to 100 ng/mL, or about 20 to 50 ng/mL, or about 25 ng/mL.

In one example, the population of cells enriched for EPCs described herein, according to any example, are non-adherent EPCs.

In one example, the methods described herein, according to any example, are performed in vitro or ex vivo.

In one example, the EPC containing population of cells is isolated from blood.

In one example, the EPC containing population of cells is isolated from umbilical cord blood.

For example, the methods described herein according to any example, the cells are from the subject to be treated, i.e., an autologous transplant, or from a related subject of the same or unrelated species (e.g., a HLA matched subject or xenograft), i.e., an allogeneic or xenogeneic transplant.

Key to sequence listings

SEQ ID NO: 1 amino acid sequence of Homo sapiens IL-3

SEQ ID NO: 2 amino acid sequence of Homo sapiens TPO

SEQ ID NO: 3 amino acid sequence of Homo sapiens SCF

SEQ ID NO: 4 amino acid sequence of Homo sapiens Flt3L

SEQ ID NO: 5 amino acid sequence of Homo sapiens PIGF

### BRIEF DESCRIPTION OF DRAWINGS

FIG. 1 is a series of graphical representations showing that CD133+ non-adherent EPC expansion is enhanced by IL-3 and is not affected by freeze/thaw. CD133+ non-adherent EPCs isolated from a single donor were split and grown under four different condition; media alone (−), medi+PIGF, media+IL-3 and media+PIGF+IL-3. Panel A shows cell proliferation in different growth conditions at day 6 to 7 of expansion (i) and day 12 to 13 of expansion (ii). Data are expressed as the fold change from the starting number of CD133+ non-adherent EPCs normalised to media alone expansion, mean±SEM, n=5-8, * p<0.05 compared to media alone. Panel B shows the ability of CD133+ non-adherent EPCs to be expanded after being frozen and thawed. Non-adherent EPCs were isolated from cord blood based on CD133 expression and expanded in media (thick black line) or frozen down in FCS 90%/DSMO 10% and placed into liquid nitrogen for at least one week before thawing and expanding in media (thin black line). Cell expansion (i) and viability (ii) were recorded for 14 days, graphs show mean±SEM, n=3.

FIG. 2 is a series of graphical representations showing that the surface expression profile of CD133+ non-adherent EPCs is not influenced by IL-3. CD133+ isolated non-adherent EPCs were expanded in CellGro media with IL-3 (dashed grey line) or without IL-3 (solid black line). Solid grey peak represents negative control. Cells were labelled with fluorescently tagged antibodies targeting haempoietic progenitor, endothelial, leukocyte and monocyte cell markers (as indicated below each graph). The ability of cells to take up DiI-Ac-LDL and FITC-labelled UEA-1 lectin were also assessed by flow cytometry. Representative donor cell line on day 9 of expansion (n=4).

FIG. 3 is a series of graphical representations showing MHC expression on non-adherent EPCs. Donor matched non-adherent EPCs, Human Umbilical Vein Endothelial Cells (HUVECs) and mature Dendridic cells (mDCs) were isolated from umbilical cords. After 7 days in culture they were labelled with antibodies against MHC class I (Panels A,C,E), MHC class II (Panels B,D,F) or an isotype control. Panels A and B show the fold change in mean fluorescence intensity (MFI) compared to the isotype control was calculated for each cell type from each donor, in addition non-donor matched mesenchymal stem cells (MSCs) were used as a comparator cell. Each triangle represents one data point, the line represents the mean. Panels C and D show the expression of MHC on each donor HUVEC and mDCs were normalised to the donor matched non-adherent EPC, n=4-9. Panels E and F) show donor matched EPCs and HUVEC and unmatched MSCs were cultured with 10 ng/ml of TNFα (grey bars) or IFNγ (black bars) for 48 hrs prior to antibody labelling, the change in MHC expression was normalised to the respective cell type grown in the absence of cytokine (no treatment (NT), white bars), n=3-6. All bar histograms show mean±SEM. * indicates p<0.05 compared to non-adherent EPC (A-D) or NT cells (E-F).

FIG. 4 is a series of graphical representations showing expanded non-adherent EPCs contribute to tube formation in vitro. To determine the ability of CD133+ non-adherent EPCs to contribute to endothelial tube formation, mature HUVECs were cultured in vitro on Matrigel™ for 6 hrs alone or with non-adherent EPCs expanded in the presence or absence of IL-3. The number of tubes, branches and loops (as indicated) were quantified from 3 biological replicates of n=5. Data expressed as mean±SEM. * indicates p<0.05 compared to 2×10⁴ HUVEC, # indicates a statistical difference between HUVEC+EPC and HUVEC+IL-3 EPC, p<0.05.

FIG. 5 is a graphical representation showing pro-angiogenic effects of expanded non-adherent EPCs in Matrigel™ plugs in vivo. Matrigel™ plugs containing non-adherent EPCs or PBS alone were injected into each flank of NOD/SCID mouse and removed after 13 days. Paraffin embedded plugs were sectioned and were labelled with either anti-mouse/ human CD31 or the human-specific mitochondria marker MTCO2. The number of CD31+ vessels in the tissue surrounding the Matrigel™ plug was quantified, mean±SEM is shown, n=4, * indicates p<0.05.

FIG. 6 is a series of graphical representations showing effects of intracardiac injection of non-adherent EPCs into rats with surgically-induced (acute myocardial infarction) AMI. Seven days after AMI and non-adherent EPC delivery the left ventricle ejection fraction (LVEF) shown as ejection fraction % was assessed by MRI (A) and peripheral blood was taken to measure serum creatinine (B). Data is shown as the mean±SEM, n=3-6, different letters indicate p<0.05 between groups.

FIG. 7 is a series of graphical representations showing rat gene expression in cardiac tissue 7 days post AMI. Rats given surgically-induced AMI received either PBS (grey bars) or 1×10⁶ non-adherent EPCs (black bars) via injections into the infarct site. Seven days post-surgery cardiac tissue from the infarct site was collected, RNA isolated and rat gene expression (as indicated) was analysis by real time PCR. Control heart tissue was collected from rats which had not undergone any procedure (white bars). Gene expression was normalised to housekeepers. Data is shown as the mean±SEM fold change compared to the control rats, n=3-6, different letters indicate p<0.05 between groups.

### DETAILED DESCRIPTION

### General

The term “and/or”, e.g., “X and/or Y” shall be understood to mean either “X and Y” or “X or Y” and shall be taken to provide explicit support for both meanings or for either meaning.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the present disclosure and individual examples thereof are susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples of the disclosure included herein, which are intended for the purpose of exemplification only. Functionally-equivalent products, compositions and methods are clearly within the scope of the disclosure and examples thereof, as described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al., (editors) Current Protocols in Immunology, John Wiley & Sons (including all updates until present).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The steps, features, integers, compositions and/or compounds disclosed herein or indicated in the specification of this application individually or collectively, and any and all combinations of two or more of said steps or features.

### Selected Definitions

As used herein, the term “culture” shall be understood to mean the maintenance and growth of cells of a multicellular organism outside the body in specifically designed containers and with precise conditions of temperature, humidity, atmosphere and nutrients free from contamination.

Reference here to “culturing an EPC-containing population” necessarily encompasses culturing the population of cells enriched for EPCs.

As used herein, the term “endothelial progenitor cell” or “EPC” shall be understood to mean a cell of the endothelial lineage capable of differentiating into a mature endothelial cell, for example, a blood vessel endothelial cell. This term does not include embryonic stem cells or induced pluripotent cells (which are capable of differentiating into endothelium). Exemplary EPCs are monocytic EPCs or hemangioblastic EPCs. Exemplary EPCs express at least CD31. Alternatively, or in addition, the EPCs express at least CD133. EPCs may also express CD1a and/or CD45 and/or CD31 and/or VEGFR2. Alternatively, or in addition, an EPC does not express significant or above background levels of CD144 and/or vWF and/or eNOS and/or Tie2. Alternatively or in addition, EPCs produce pro-angiogenic factors, e.g., hepatocyte growth factor and/or insulin-like growth factor-1 and/or basic fibroblast growth factor and/or VEGF. In one example, the EPCs do not adhere to tissue culture plastic-ware, optionally plastic-ware coated with extracellular matrix or a component thereof (e.g., fibronectin). Therefore, in one example the EPCs used in the present disclosure are “non-adherent EPCs”. In one example, the EPCs are non-adherent CD133 expressing mononuclear cells. In one example, the EPCs are non-adherent DSG2+ expressing mononuclear cells. In one example, the EPCs are non-adherent CD34+ and DSG2+ expressing mononuclear cells that are CD45 dim.

The term “endothelium” or “endothelial cell” shall be understood to mean a tissue or cell that lines tissues of the circulatory system.

The term “EPC-associated condition” shall be taken to encompass any disease or disorder or state in which modulation of EPC numbers and/or activity may provide a beneficial effect and/or characterized by excessive or insufficient EPC numbers and/or activity. Exemplary conditions are described herein and are to be taken to apply mutatis mutandis to those examples of the disclosure relating to diagnosis/prognosis/treatment/prophylaxis of an EPC-associated condition. In one example, an EPC-associated condition is characterized by insufficient EPC numbers and/or activity. Exemplary conditions include cardiovascular disease, autoimmune conditions (e.g., rheumatoid arthritis, psoriatic arthritis, systemic lupus erythematosus and systemic sclerosis), antineutrophil cytoplasmic antibodies (ANCA)-associated vasculitis, ischemia (including ischemia resulting from a transplant) and testicular necrosis. In another example, the condition is associated with excessive EPC numbers and/or activity (including excessive neovascularization). Exemplary conditions include cancer (including solid tumors, leukemias, lymphoma, melanoma, glioma, breast cancer, colonic cancer, gastric cancer, esophageal cancer, renal cell cancer, ovarian cancer, cervical cancer, carcinoid cancer, testicular cancer, prostate cancer, head and neck cancer and hepatocellular carcinoma), cancer metastasis, cancer neovascularization, autoimmune disease (including psoriasis), nephropathy, retinopathy, preeclampsia hepatitis, sepsis and macular degeneration.

As used herein, the term “EPC activity” will be understood to encompass any function that is characteristic of an EPC and includes any one or more of the following:
a. Uptake of diacetylated LDL (Dil-Ac-LDL);
b. Binding of Ulex europaeus I lectin;
c. Labeling with antibodies that bind to CD34, CD133 and VEGF-R2;
d. Ability to form tubes in vitro either alone or in conjunction with mature endothelial cells;
e. Migration towards angiogenic factors (such as VEGF) in vitro or in vivo;
f. Secretion of angiogenic factors (such as VEGF, hepatocyte growth factor, granulocyte-colony stimulating factor, macrophage migration inhibitory factor, interleukin 8);
g. Ability to induce neovascularization in vivo; and
h. Ability to form colony forming units (CFUs).

Assays for determining EPC activity are known in the art and/or described in more detail herein.

As used herein, the term “expand” or “expanded” in the context of a cell population shall be taken to encompass an increase in the number or percentage of EPCs that is greater than the number or percentage of EPCs in the starting population. For example an expanded population of EPCs has at least about a 50 fold, or at about least 100 fold, or at least about a 150 fold, or at least about a 200 fold, or at least about a 250 fold, or at least about a 300 fold increase in EPCs.

As used herein, the term “enriched” or “enrich” in the context of a cell population shall be taken to encompass a population of cells comprising EPCs, in which the number or percentage of EPCs is greater than the number or percentage in a naturally occurring cell population. For example, a population enriched in EPCs is made up of at least about 0.02% of said cells, or at least about 0.05% of said cells or at least about 0.1% of said cells or at least about 0.2% of said cells or at least about 0.5% of said cells or at least about 0.5% of said cells or at least about 0.8% of said cells or at least about 1% of said cells or at least about 2% of said cells or at least about 3% of said cells or at least about 4% of said cells or at least about 5% of said cells or at least about 10% of said cells or at least about 15% of said cells or at least about 20% of said cells or at least about 25% of said cells or at least about 30% of said cells or at least about 40% of said cells or at least about 50% of said cells or at least about 60% of said cells or at least about 70% of said cells or at least about 80% of said cells or at least about 85% of said cells or at least about 90% of said cells or at least about 95% of said cells or at least about 97% of said cells or at least about 98% of said cells or at least about 99% of said cells.

As used herein, the term “IL-3” shall be taken to mean interleukin-3. Interleukin-3 (IL-3) also known as multipotential colony-stimulating factor is a cytokine, specifically an interleukin involved in cell proliferation and differentiation. Interleukin-3 (IL-3) is a hematopoietic growth factor which has the property of being able to promote the survival, growth and differentiation of hematopoietic cells. In one example, IL-3 is of human, animal origin or is recombinant. In another example IL-3 is a fusion protein. The IL-3 protein sequence is exemplified in SEQ ID NO:1, NCBI Ref Seq ID numbers NM_000588.3 and NP 000579.2. In one example, the IL-3 protein has at least 70% identity to SEQ ID NO: 1.

As used herein, the term “TPO” shall be taken to mean thrombopoietin. Thrombopoietin (TPO or THPO) also referred to in the literature as thrombocytopoiesis stimulating factor (TSF), megakaryocyte colony-stimulating factor (MK-CSF), megakaryocyte-stimulating factor and megakaryocyte potentiator is encoded by the THPO gene in humans. TPO is a glycoprotein hormone that regulates the production and differentiation of megakaryocytes which are important in the production of platelets. The TPO protein sequence is exemplified in SEQ ID NO:2, and NCBI Ref SEQ ID numbers NM_000460.3 and NP_000451.1. In one example, the TPO protein has at least 70% identity to SEQ ID NO: 2.

As used herein, the term “SCF” shall be taken to mean stem cell factor. SCF is a cytokine that binds to CD117. SCF exists as both a transmembrane and a soluble protein and plays an important role in haematopoiesis in embryonic development. The SCF protein sequence is exemplified in SEQ ID NO:3, and Genbank accession AAA85450.1. In one example, the SCF protein has at least 70% identity to SEQ ID NO: 3.

As used herein, the term “Flt3L” shall be taken to mean Flt3-ligand. Flt3-ligand is a cytokine encoded by the FLT3LG gene. The Flt3L protein sequence is exemplified in SEQ ID NO: 4, and Genbank accession AAA19825.1. In one example, the SCF protein has at least 70% identity to SEQ ID NO: 4.

As used herein, the term “PIGF” shall be taken to mean placental growth factor. Placental growth factor (PIGF, P1GF or PGF) binds the VEGF receptor and plays a role in angiogenesis. The Flt3L protein sequence is exemplified in SEQ ID NO: 5 and Genbank accession number AAD30179.1. In one example, the PIGF protein has at least 70% identity to SEQ ID NO: 5.

As used herein, the term “CD133+” shall be taken to mean a cell that expresses a detectable level of CD133 polypeptide, polynucleotide of fragment thereof

As used herein, the term “CD34+” shall be taken to mean a cell that expresses a detectable level of CD34 polypeptide, polynucleotide of fragment thereof

As used herein, the term “DSG2+” shall be taken to mean a cell that expresses a detectable level of DSG2+ polypeptide, polynucleotide of fragment thereof

As used herein, the term “CD45 dim” shall be taken to mean a cell that has low expression of CD45.

As used herein, the term “CD34+, CD45 dim DSG2+ cell” shall be taken to mean a cell that expresses CD34+ and DSG2+ and has low expression of CD45.

As used herein, the terms “preventing”, “prevent” or “prevention” include administering a therapeutically effective amount of an inhibitor(s) and/or agent(s) described herein sufficient to stop or hinder the development of at least one symptom of a specified disease or condition.

As used herein, the term “subject” shall be taken to mean any subject comprising EPCs, for example, a mammal. Exemplary subjects include but are not limited to human, primate, livestock (e.g. sheep, cow, horse, donkey, pig), companion animals (e.g. dogs, cats), laboratory test animals (e.g. mice, rabbits, rats, guinea pigs, hamsters), captive wild animal (e.g. fox, deer). For example, the mammal is a human or primate. In one example, the mammal is a human.

As used herein, the terms “treating”, “treat” or “treatment” include administering a therapeutically effective amount of a compound described herein sufficient to reduce or eliminate at least one symptom of a specified disease or condition.

As used herein, the term “neovascularization” relates to the formation of functional microvascular networks. Various disorders are associated with insufficient neovascularization, e.g., ischemia or aberrant angiogenesis/vasculogenesis, e.g., cancer. In this regard, the skilled artisan will be aware that neovascularization encompasses angiogenesis and vasculogenesis. Angiogenesis is the growth of new blood vessels from pre-existing vessels. Angiogenesis can take two forms, i.e., sprouting angiogenesis is the formation of new vessels toward an angiogenic signal, and intussusceptive angiogenesis is the process by which a blood vessel is split into two new vessels. In contrast, vasculogenesis is the de novo formation of blood vessels by tissue resident endothelial progenitor cells (EPCs).

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of the population of cells enriched for EPCs, rather the present disclosure encompasses any amount of the population of cells enriched for EPCs (s) sufficient to achieve the stated result in a subject.

### Methods

### Isolation or Enrichment of Cells

One exemplary approach to enrich for the desired cells (e.g. DSG2+ or CD133+) is magnetic bead cell sorting (MACS) or any other cell sorting method making use of magnetism, e.g., Dynabeads®. A conventional MACS procedure is described by Miltenyi et al. (1990). In this procedure, cells are labelled with magnetic beads bound to an antibody or other compound that binds to a cell surface marker or protein and the cells are passed through a paramagnetic separation column or exposed to another form of magnetic field. Cells that are magnetically labelled are trapped in the column; cells that are not pass through. The trapped cells are then eluted from the column.

The MACS technique is equally applicable to negative selection, e.g., removal of cells expressing an undesirable marker, i.e., undesirable cells. Such a method involves contacting a population of cells (for example, as described herein the EPC containing population of cells or a population of cells enriched for EPCs) with a magnetic particle labelled with a compound that binds to a cell surface marker expressed at detectable levels on the undesirable cell type(s). Following incubation, samples are washed and resuspended and passed through a magnetic field to remove cells bound to the immunomagnetic beads. The remaining cells depleted of the undesirable cell type(s) are then collected.

In another example, a compound that binds to a protein or cell surface marker is immobilized on a solid surface and a population of cells is contacted thereto. Following washing to remove unbound cells, cells bound to the compound can be recovered, e.g., eluted, thereby isolating or enriching for cells expressing the protein to which the compound binds. Alternatively, cells that do not bind to the compound can be recovered if desired.

In a further example, cells are isolated or enriched using fluorescence activated cell sorting (FACS). FACS is a known method for separating particles, including cells, based on the fluorescent properties of the particles and described, for example, in Kamarch (1987). Generally, this method involves contacting a population of cells with compounds capable of binding to one or more proteins or cell surface markers, wherein compounds that bind to distinct markers are labelled with different fluorescent moieties, e.g., fluorophores. The cells are entrained in the center of a narrow, rapidly flowing stream of liquid. The flow is arranged so that there is a separation between cells relative to their diameter. A vibrating mechanism causes the stream of cells to break into individual droplets. The system is adjusted so that there is a low probability of more than one cell being in a droplet. Just before the stream breaks into droplets the flow passes through a fluorescence measuring station where the fluorescent character of interest of each cell is measured, e.g., whether or not a labeled compound is bound thereto. An electrical charging ring is placed at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge, e.g., into one container if a labelled compound is bound to the cell and another container if not. In some systems the charge is applied directly to the stream and the droplet breaking off retains charge of the same sign as the stream. The stream is then returned to neutral after the droplet separates.

### Cell Culture

Cell cultures can be incubated at about 37° C. in, for example, in a humidified incubator or bioreactor. Exemplary temperatures for cell culture include, for example, at 35-37° C., or 35-39° C., or 30-39° C., or 25-39° C., or 20-39° C,or 15-40° C., or 10-45° C. Cell culture conditions can vary considerably for the cells of the present disclosure. For example, the cells are maintained in an environment suitable for cell growth, e.g., comprising 5% O₂, 10% CO₂, 85% N₂ or comprising 10% CO₂ in air.

Exemplary media for culture comprises salts, sugars, amino acids, vitamins, buffers, phenol-red pH indicator, L-glutamine, β-mercaptoethanol, human albumin, rh insulin (e.g. expressed in yeast) at an osmolality of 290 -350 (mOsm/kg H₂O) and a pH of 7.2 to 7.5 and, for example, does not comprise serum.

Exemplary amino acids include alanine, histidine, arginine, isoleucine, leucine, lysine, methionine, phenylalanine, threonine, tryptophan, valine glutamine, cystine, serine and proline.

Exemplary salts include: calcium chloride, magnesium chloride and sodium bicarbonate,

Exemplary sugars include: glucose, galactose and fructose.

Exemplary vitamins include vitamin B12, vitamin A and vitamin E.

In some examples cells are cultured in media without serum. Exemplary media include CellGro Stem Cell Growth Medium (SCGM). For example, SCGM comprises; salts, sugars, amino acids, vitamins, buffers, phenol-red pH indicator, L-glutamine, β-mercaptoethanol, human albumin, rh insulin (e.g. expressed in yeast) at an osmolality of 290 -350 (mOsm/kg H₂O) and a pH of 7.2 to 7.5.

In some examples cells can be maintained in Dulbecco's Minimal Essential Medium (DMEM) or any other appropriate cell culture medium known in the art, e.g., as described above. Other appropriate media include, for example, MCDB, Minimal Essential Medium (MEM), IMDM, and RPMI. Additional suitable media for culturing EPCs include endothelial growth media, such as EGM-2 plus Bullet kit (available from Lonza Group Ltd).

In some examples cells are cultured in the presence of one or a combination of the following factors: IL-3, TPO, Flt3L, SCF, PIGF, Pen/Strep.

In one example, TPO stock is reconstituted in 10 μL of 10 mM sodium citrate, pH 3.0 made up to a total volume of 5 mL with incomplete CellGro SCGM media. Reconstituted aliquots have a concentration of 1 μg/mL and are stored at −80° C. Once thawed TPO can be stored for 1 week at 4° C. In one example, the final concentration in media is 10 ng/mL, in this example 10 μL of stock is added per mL of media.

In one example, Flt3L stock is reconstituted in PBS/BSA to a concentration of 100 μg/mL and aliquots are stored at −80° C. Flt3L aliquots, once thawed are stored at 4° C. for up to one month. In some examples the final Flt3L concentration in media is 40 ng/mL.

In one example, SCF stock is reconstituted in PBS/BSA to a concentration of 20 μg/mL and aliquots are stored at −80° C. Once thawed SCF aliquots are stored at 4° C. for one month. Final concentration in media=40 ng/mL, therefore add 2 μL of stock / mL of media.

In one example, PIGF is at a concentration of 10 μg/mL. Once thawed PIGF can be stored for up to 1 month at 4° C. In one example, the final concentration of PIGF is 25 ng/mL, in this instance 2.5 μL of stock is adder per mL of media.

In one example, IL-3 is added at a concentration of 1 μL/mL of media, for a final concentration in the media of 25 ng/mL.

In some examples cells may be cultured in the presence of antibiotics. Exemplary antibiotics include penicillin/streptomycin, amphotericin, nystatin, gentamicin and kanamycin. In one example, cells are culture in the presence of penicillin/streptomycin for example, at a concentration of 0.5% (v/v).

In another example, the cells are cultured in suspension, i.e., without adhering to tissue culture plastic-ware or an extracellular matrix or components thereof. In this regard, the inventors have clearly exemplified culturing of EPCs in suspension culture.

In one example, the cells are cultured in cell culture plates, cell culture flasks, tissue culture containers or in a bioreactor. For example, cells may be cultured in a bioreactor of at least about 1 L, or at least about 2 L, or at least about 5 L, or at least about 10 L, or at least about 50 L, or at least about 100 L, or at least about 500 L, or at least about 1000 L, or at least about 2000 L, or at least about 3000 L, or at least about 5000 L.

### In Vitro Assays of EPC Activity

An exemplary in vitro method for determining EPC activity is, for example, a CFU assay in which cells are cultured on an extracellular matrix and the ability to form clonal colonies is determined. For example, EPCs are cultured for several days, e.g., at least 2 or 3 or 4 or 5 or 6 or 7 days in a suitable culture medium and the number of cell colonies adhering to the chamber in which the cells are cultured are counted. Optionally, the chamber is coated with extracellular matrix or a component thereof. Functional EPCs will be capable of forming colonies, with each colony representing a CFU. When assessing the effect of a reduction in the amount of colonies (i.e., CFUs) in the presence of the compound compared to the number of colonies (CFUs) in the absence of the compound indicates that the compound inhibits or reduces EPC activity.

Other assays include, for example, migration assays, in which the ability of an EPC to migrate in vitro to an angiogenic compound, such as, VEGF. For example, a chamber comprising a porous membrane is coated with an extracellular matrix or component thereof and EPCs cultured in the chamber. The chamber is inserted into another chamber comprising an angiogenic factor, e.g., VEGF and the cells maintained for a time sufficient for the EPCs to migrate through the pores (e.g., 4-6 hours or 1-2 days). Cells having EPC activity migrate towards the angiogenic factor and are detectable in the chamber comprising the angiogenic factor.

Other assays include those involving culturing cells and determining those capable of uptake of acetylated-LDL and/or that bind to Ulex europaeus I lectin. In such assays, cells are cultured in the presence of labelled acetylated LDL (e.g., 1,1′-dioctadecyl-3,3,3′,3-tetramethyl-indocarbocyanine perchlorate (Dil)-Ac-LDL) and/or Ulex europaeus lectin (e.g., labelled with a detectable compound). Cells that take up acetylated LDL and/or bind to Ulex europaeus lectin are considered to have EPC activity. For example, EPCs take up acetylated LDL and bind to Ulex europaeus lectin. A compound or cellular pharmaceutical compositions that inhibits or reduces EPC activity reduces uptake of acetylated LDL and/or binding of Ulex europaeus lectin.

A further method for assessing EPC function is a tube formation method. In such a method, cells are cultured in a tissue culture chamber, e.g., coated with extracellular matrix or a component thereof. Cells are cultured for a sufficient period to form tubes (e.g., 1-6 days) and the tissue culture chambers observed, using microscopy. Tubes are observed between two discrete cells or clusters thereof. Tube formation is indicative of EPC activity.

Alternatively, or in addition, EPCs function is assessed by detecting secretion of an angiogenic factor, e.g., VEGF, hepatocyte growth factor, granulocyte-colony stimulating factor, macrophage migration inhibitory factor and interleukin 8. For example, cells are cultured for a suitable period of time (e.g., 1-6 days) and the level of angiogenic factors in culture medium determined using, for example, an ELISA or a FLISA. Secretion of higher levels of angiogenic factors than a non-EPC endothelial cell indicates EPC activity.

### In Vivo Assays of EPC Function

In another example, a population of cells (for example, the EPC containing population of cells or a population of cells enriched for EPCs) isolated by a method as described herein according to any example is determined by administering the cells to an animal model of a condition associated with EPCs. For example, the cells are administered to an animal lacking EPCs e.g., as a result of myeloablation or mice having defects in angiogenesis (e.g., Idl-deficient mice; Lyden et al., 2001). Cells that facilitate or contribute to neovascularization are considered to have EPC function. Alternatively, or in addition, cells are administered to an animal model of ischemia, such as, hind-limb ischemia and/or cardiovascular ischemia and/or stroke and the effect of the cells on neovascularization is determined. Exemplary models are described, for example, in Couffinhal et al. (1998) or Carmeliet et al. (2000).

In another example, EPC activity is assessed by mixing EPCs with matrigel to form a plug and administering the plug subcutaneously to a non-human mammal, e.g., a mouse. After a sufficient period, e.g., about 7 days, the plug is removed and analyzed microscopically for evidence of formation of blood vessels, i.e., neovascularization. An exemplary method is described in Bagley et al., (2003).

Alternatively, or in addition, a cell population or cellular pharmaceutical compositions is administered to an animal model of angiogenesis or insufficient neovascularization and the level of blood vessel formation determined. For example, a cell population or cellular pharmaceutical compositions is administered to a test subject. The presence/absence and/or extent of neovascularization is assessed and compared to subjects to which the cells or pharmaceutical population has not been administered. For example, the amount of vascularization is determined in the tumor test tissue to determine a cell population or cellular pharmaceutical compositions that promote or suppress neovascularization.

### Cellular Pharmaceutical Compositions

In one example, of the present disclosure EPCs and/or progeny cells thereof are administered in the form of a composition. For example, such a composition is a pharmaceutical composition and comprises a pharmaceutically acceptable carrier and/or excipient.

Suitable carriers for this disclosure include those conventionally used, e.g., water, saline, aqueous dextrose, lactose, Ringer's solution, a buffered solution, hyaluronan and glycols which are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay an EPC-associated condition.

In one example, the carrier or excipient provides a buffering activity to maintain the cells at a suitable pH to thereby exert a biological activity, e.g., the carrier or excipient is phosphate buffered saline (PBS). PBS represents an attractive carrier or excipient because it interacts with cells minimally and permits rapid release of the cells, in such a case, the composition of the disclosure may be produced as a liquid for direct application to the blood stream or into a tissue or a region surrounding or adjacent to a tissue, e.g., by injection.

EPCs and/or progeny cells thereof can also be incorporated or embedded within scaffolds that are recipient-compatible and which degrade into products that are not harmful to the recipient. These scaffolds provide support and protection for cells that are to be transplanted into the recipient subjects. Natural and/or synthetic biodegradable scaffolds are examples of such scaffolds. Other suitable scaffolds include polyglycolic acid scaffolds, e.g., as described by Vacanti, et al. (1988); Cima, et al. (1991); Vacanti, et al. (1991); or synthetic polymers such as polyanhydrides, polyorthoesters, and polylactic acid.

For example, the composition comprises an effective amount or a therapeutically or prophylactically effective amount of cells. For example, the composition comprises about 1×10⁵ EPCs/kg to about 1×10⁹ EPCs/kg or about 1×10⁶ EPCs/kg to about 1×10⁸ EPCs/kg or about 1×10⁶ EPCs/kg to about 1×10⁷ EPCs/kg. The exact amount of cells to be administered is dependent upon a variety of factors, including the age, weight, and sex of the patient, and the extent and severity of the EPC-associated condition.

The cellular compositions of this disclosure can be administered to the subject by any recognized methods, either systemically or at a localized site. In one example, the most convenient time to administer the cells to improve grafting is during the time of surgery. To treat an autoimmune disease, the composition can be administered at the onset of symptoms and/or following onset of symptoms or even prior to the onset of symptoms (e.g., following detection of an autoimmune response). To keep the cells at the site until completion of the surgical procedure, it is convenient to administer the cells in a pharmaceutically compatible artificial gel, or in clotted plasma, by utilizing any other known controlled release mechanism (see above), or immobilized on a solid or semi-solid support. When less invasive procedures are desired, the composition can be injected at a desired location through a needle. For deeper sites, the needle can be positioned using endoscopic ultrasound techniques, radioscintigraphy, or some other imaging technique, alone or in combination with the use of an appropriate scope or cannula. For such applications, the cell population is conveniently administered when suspended in isotonic saline or a neutral buffer.

In one example, a cellular composition of the present disclosure is administered together with an agent that enhances endothelialization, such as, VEGF. The cells and the agent can be administered in the same composition and/or can be administered separately.

As discussed herein, EPCs and/or compositions that bind to EPCs can be immobilized on a solid or semi-solid matrix prior to administration to a subject. Such matrices are useful for, for example, forming vascular grafts that are endothelialized, thereby reducing the risk of thrombosis. Exemplary matrices will be apparent to the skilled artisan and include hydrogel materials, blends of hydrophilic and hydrophobic polymers such as polyethylene glycol (PEG) and d,l-polylactic acid (d,l-PLA), polyester and polytetrafluoroethyle.

### Method of Treatment

Exemplary EPC-associated conditions for treatment using a method, as described herein according to any example of the disclosure, include the following: a cardiovascular disease (including coronary artery disease or dysfunctional bicuspid aortic valve) or cerebrovascular disease which can be diagnosed/prognosed by detecting reduced levels of EPCs; on autoimmune disease, e.g., rheumatoid arthritis, systemic lupus erythematosus, diabetes (e.g., type 1 diabetes) or systemic sclerosis, e.g., more than five years after onset which can be diagnosed/prognosed by detecting reduced or increased levels of EPCs; ischemia, e.g., stroke, which can be diagnosed/prognosed by detecting reduced levels of EPCs; sepsis, which can be diagnosed by detecting reduced levels of EPCs.

The skilled artisan will appreciate that methods described herein for isolating an EPC also provide the basis for increasing EPC numbers in a subject, e.g., by adoptive transfer or cell therapy. Increasing EPCs numbers is useful for, for example, treating or preventing a condition associated with reduced EPC numbers and/or inducing neovascularization, e.g., to improve grafting or wound healing or reduce the effects of ischemia and/or to reduce hypertension and/or to improve healing of bone defects. Accordingly, another example of the present disclosure provides a method of treating or preventing a condition associated with reduced EPCs or activity, treating or preventing a condition associated with insufficient neovascularization and/or improving grafting and/or improving wound healing in a subject, said method comprising:(i) isolating a population enriched for EPCs by performing a method of the disclosure; and (ii) administering the cells at (i) to the subject.

In another example, the disclosure provides a method of treating or preventing a condition associated with reduced EPC numbers or activity, treating or preventing a condition associated with insufficient neovascularization and/or improving grafting and/or improving wound healing in a subject, the method comprising administering a composition comprising a population of cells enriched for EPCs of the disclosure.

In the situation of a graft, e.g., a blood vessel graft, the cells can be administered immobilized on a solid support or semi-solid support, e.g., in the form of a vascular graft.

In one example, the subject suffers from or is at risk of developing a condition associated with reduced EPC numbers and/or activity and/or a condition associated with insufficient neovascularization and/or requires a graft or has undergone grafting and/or requires improved wound healing.

### Kits

The present disclosure also provides kits comprising media and instructions for producing a population of EPC cells of the present disclosure or reagents for isolating the populations of EPC cells of the present disclosure or therapeutic and prophylactic kits comprising the population of EPC cells or a pharmaceutical composition of cells for use in detection/isolation/diagnostic/prognostic/treatment/prophylactic methods. Such kits will generally contain, in suitable container means, a media or population of EPC cells of the present disclosure. The kits may also contain other compounds, e.g., for detection/isolation/diagnosis/imaging or combined therapy. For example, such kits may contain any one or more of a range of anti-inflammatory drugs and/or chemotherapeutic or radiotherapeutic drugs; anti-angiogenic agents; anti-tumor cell antibodies; and/or anti-tumor vasculature or anti-tumor stroma antibodies or coaguligands or vaccines.

In one example, the kit comprising the medium described herein, according to any example, for culturing EPCs of the disclosure, optionally, packaged with instructions for use in a method as described herein.

In another example, the kit is for isolating and/or expanding the EPCs of the disclosure, optionally, packaged with instructions for use in a method as described herein. In one example, the kit contains a serum free medium and one or more of IL-3, TPO, Flt3L, SCF and PIGF. In one example, the kit comprises IL-3. In one example, the kit may include agents for selection of EPCs that are either CD133+, DSG2+ or CD34+, DSG2+ and CD45dim.

In a further example, the kit is for treatment or prevention of an EPC-associated condition. In such kits, the population of cells enriched for EPCs, or the pharmaceutical composition of cells enriched for EPCs may be provided in solution. As discussed above, the kit may also comprise additional therapeutic or prophylactic compounds.

The present disclosure includes the following non-limiting examples.

### EXAMPLES

### Example 1

### Protocol for Human EPC Isolation from Umbilical Cord Blood and Expansion

Umbilical cord blood was collected from healthy, consenting women undergoing elective caesarean sections in cord blood bags (MacroPharma, Mouvaux, France). Cord blood was diluted 1:1 (in sterile 50 ml tubes) at room temperature (RT) with lx sterile phosphate buffered saline (PBS). In a new 50 ml tube 15 ml Lymphoprep™ (Axis-Shield, Oslo, Norway) at RT was added and 35 ml of PBS diluted cord blood carefully layered onto the Lymphoprep™. Cells were centrifuged at 1,800 rpm (Eppendorf 5810R centrifuge with A-4-81 rotor) for 30 min with no brake and extremely low acceleration at RT. The mononuclear cell layer (MNC) was collected and placed in a new 50 mL tube. Next, cells were diluted with 25 mL human umbilical vein endothelial (HUVE) media (Media 199 (Sigma-Aldrich St. Louis Mo.) containing; 20% foetal calf serum (FCS; Hyclone, Logan Utah) endothelial growth factor supplement (BD BD BioSciences, San Jose, Calif.), 1.5% sodium bicarbonate, 2% HEPES buffer solution, penicillin streptomycin, sodium pyruvate (Gibco Invitrogen, Gaithersburg, Md.), heparin and non-essential amino acids (Sigma-Aldrich) and centrifuged at 1,500 rpm for 5 min at RT. The supernatant was removed and cells resuspended in 20 mL HUVE media. The HUVE media wash was repeated a total of three times. Next, cells were resuspended in 10 mL of 5% normal mouse serum (Abcam, Cambridge, England) in HUVE media for 10 min followed by centrifugation at 1,500 rpm for 5 min at RT. The supernatant was aspirated and ≦1×10⁸ cells resuspended in 500 μL human FcR blocking reagent (Miltenyi Biotec, Bergisch Gladbach, Germany) and optionally 100 μL CD133+ microbeads (Miltenyi Biotec) and incubated for 30 minutes at 4° C. in the dark. EPCs were isolated selected by either CD133+ or selection of CD34+, CD45 dim and DSG2+ positive cells. CD133+ cells were selected using the AutoMacsPro (Miltenyi Biotec) as per standard operating procedures. Cells were transferred to microtubes and centrifuged at 3,000 rpm for 3 min at 4° C. and the supernatant removed. Cells were then resuspended in 3 mL sterile MACS buffer and inserted into the AutoMacsPro in a chilled AutoMac rack. Alternatively cells can be separated by FACS using the same protocol except selection for CD34+, CD45 dim and DSG2+. Both selection methods produce non-adherent and adherent cell populations.

Following FACS cells were counted and centrifuged at 1,500 rpm for 5 min at RT. Cells were then resuspended with 10 ng/mL rh Thrombopoietin (TPO, Sigma-Aldrich), 40 ng/ml rhStem Cell Factor (SCF) and 40 ng/mL rhFtl3L (both R&D Systems, Minneapolis, Minn.), here on in referred to as CellGro media, as a concentration of 0.5×106 cells per mL. In some experiments IL-3 (20 ng/mL, in house) and placental growth factor (PIGF, 25 ng/ml R&D Systems, Minneapolis, Minn.) were added to the media.

Cells were then cultured for up to 14 days. Media was replenished but not removed and cells counted every 2-3 days using trypan blue and a haemocytometer as per standard protocols. The media was “topped up” to return the concentration of cells at 500,000 per mL. The old media was not removed from the cell culture. After topping of the media the required amount of cytokines/growth factors were added so the culture had the following concentrations: TPO 10 ng/mL, Flt3L 40 ng/mL, SCF 40 ng/mL, PIGF 25 ng/mL, IL-3 25 ng/mL and pen/strep 0.5%. The pen/strep was only added on the first media top up. Non-adherent cells were harvested by aspirating the culture media, leaving the adherent cells in the culture vessel.

### Example 2

### Isolation and Expansion of a Non-Adherent CD133+ Endothelial Progenitor Cells is Enhanced by IL-3

### 2.0 Methods:

### 2.1 Isolation and Culture of CD133⁺ Non-Adherent EPCs

Umbilical cord blood was collected from healthy, consenting women undergoing elective caesarean sections in cord blood bags (MacroPharma, Mouvaux, France). Blood was diluted 1:1 in sterile phosphate buffered saline (PBS). Mononuclear cells (MNCs) were isolated using Lymphoprep™ (Axis-Shield, Oslo, Norway). Mononuclear cells were washed in human umbilical vein endothelial (HUVE) media [Media 199 (Sigma-Aldrich St. Louis, Mo.); containing 20% FCS (Hyclone, Logan, Utah), endothelial growth factor supplement (BD BioSciences, San Jose, Calif.), 1.5% sodium bicarbonate, 2% HEPES buffer solution, penicillin streptomycin, sodium pyruvate (Gibco Invitrogen, Gaithersburg, Md.), heparin and non-essential amino acids (Sigma-Alrich)]. Cells were blocked with 10 ml of 5% normal mouse serum (Abcam, Cambridge, England) in HUVE media for 10 min, washed in HUVE media and blocked with 100 μl of human FcR blocking reagent (Miltenyi Biotec, Bergisch Gladbach, Germany) prior to an incubation with 100 μl of CD133⁺ microbeads (Miltenyi Biotec) for 30 minutes at 4° C. Cells were washed again in HUVE media and CD133⁺ cells isolated using the AutoMacsPro (Miltenyi Biotec) as per manufacturer's instructions. CD133⁺ cells were resuspended at a concentration of 0.5-1×10⁶ cells/ml in CellGro SCGM (CellGenix GmbH, Freiburg, Germany) supplemented with 10 ng/ml Thrombopoietin (TPO, Sigma-Alrich), 40 ng/ml stem cell factor (SCF) and 40 ng/ml Ftl3L (both R&D Systems, Minneapolis, Minn.), here on in referred to as CellGro media. In some experiments IL-3 (20 ng/ml) and placental growth factor (PIGF, 25 ng/ml, R&D Systems, Minneapolis, Minn.) were added to the media. Cells were kept under continuous culture conditions where cells were counted using trypan blue and a haemocytometer and CellGro media added to adjust the cell concentration back to 0.5×10⁶ cells/ml.

### 2.2 Freezing and Thawing of Non-Adherent EPCs

CD133 isolated Non-adherent EPCs were suspended at 1×10⁶ cells/ml at 4° C. in cryopreservation media [90% FCS (Hyclone) and 10% DSMO (Sigma-Alrich)] in a Cyto.S™ vial (Greiner Bio-One GmbH, Frickenhausen, Germany) and placed in a Nalgene Mr. Frosty™ freezing container (Thermo Fisher Scientific, Waltham, Mass.) for 24 hrs and −80° C. before being transferred to liquid nitrogen. To thaw non-adherent EPCs, the vial was taken from liquid nitrogen and placed in a 37° C. water bath until only small ice crystals were present. Pre-warmed HUVE media (10 ml) was added drop wise and cells were spun down at 300 g for 5 minutes. Media was aspirated and the pellet resuspended in CellGro media to 0.5×10⁶ cells/ml and placed into a cell culture plate.

### 2.0 Results:

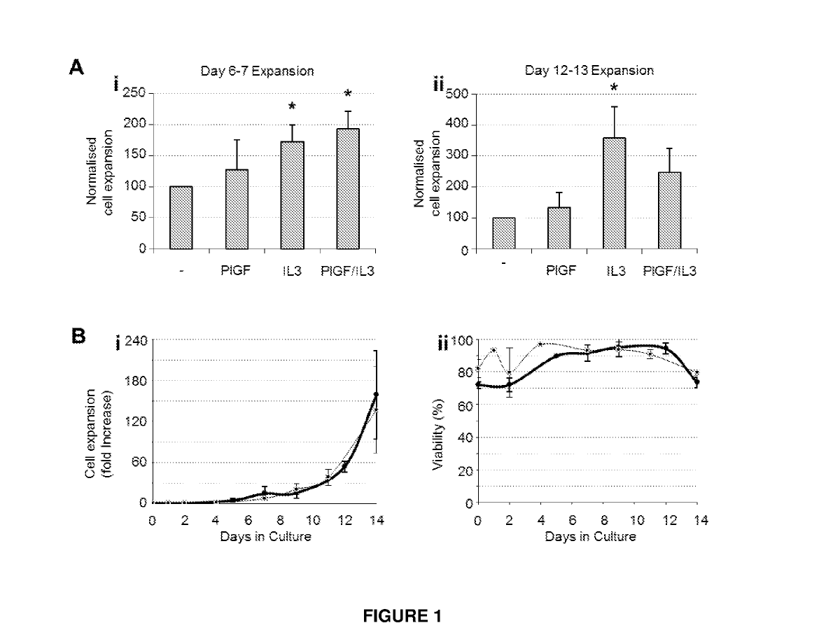

CD133+ cells were isolated from umbilical cord blood via magnetic sorting and cultured in CellGro SCGM media supplemented with TPO, Flt3L and SCF (CellGro media). After 6-7 days of culture non-adherent EPCs had expanded 14.6±45.6 fold increase at day 6-7 of culture and a fold increase of 98.6±35 at day 12-13 of culture (mean fold expansion±SEM, data not shown). PIGF and IL-3 have been shown to have proangiogenic properties (Autiero et al., 2003; Li et al., 2003; Li et al., 2006), and thus in an attempt to enhance the growth rate of CD133+ non-adherent EPCs or change their phenotype to more EC-like, PIGF (25 ng/ml) and I-L3 (20 ng/ml), either alone or in combination, were added to CellGro media. The inclusion of IL-3 into the media significantly enhanced the expansion of the non-adherent EPCs both at days 6-7 (FIG. 1Ai) and 12-13 (FIG. 1Aii) compared to donor matched cells grown in CellGro media. At day 12-13 of culture the mean fold increase of non-adherent EPCs expanded in media containing IL-3 was 267±68.

Addition of PIGF did not increase the expansion rate of non-adherent EPCs at either day 6-7 or 12-13 of culture. PIGF and IL-3 in combination did not increase the expansion rate beyond that seen in IL-3 alone, indicating that the addition of PIGF to the expansion media had no effect on cell growth and that IL-3 and PIGF do not act additively or synergistically to increase non-adherent EPC expansion (FIG. 1A).

To determine if CD133+ non adherent EPCs could be successfully expanded post freeze/thaw non adherent EPCs were expanded in CellGro media either directly after isolation from cord blood or following freezing, liquid nitrogen storage and thawing. Over the course of 14 days both the expansion rates (FIG. 1Bi) and viability (FIG. 1Bii) of both fresh and frozen expanded cells are similar.

### Example 3

### Cell Surface Phenotyping of Expanded Non-Adherent EPCs

### 3.0 Methods:

3.1 Flow Cytometric analysis of Cell Surface Protein Expression

CD133+ Non-adherent EPCs (isolated as per Example 2) were analysed for cell surface expression of various markers by flow cytometry. Cells were treated with 10 μl Human FcR block (Miltenyi Biotec) diluted in 30 μl of HUVE media. They were then incubated with a panel of mouse anti-human fluorochrome-conjugated antibodies against progenitor markers CD34, CD117, CD133, vascular markers CD31, CD144, CD146 and VEGFR2, haematopoietic markers CD14, CD38, CD45 and IL-3RA or appropriate isotype controls (all BD Bioscience, except anti-CD133 is Miltenyi Biotec) for 30 min at 4° C. Cells were washed in PBS and 5 μl of 7AAD (BD Biosciences) was added to each sample. Cells were analysed on an Accuri flow cytometer (BD Biosciences), with further analysis performed using FCS Express 4 Flow Cytometry: Research Edition (De Novo Software, Los Angeles, Calif.).

### 3.2 Up Take of Acetylated-Low Density Lipoprotein and Ulex Europaeus Lectin Binding

Cells were incubated at 37° C. for 4 hours with 10 μg/mL 1,1′-dioctadecyl-3,3,3′,3′-tetramethylindocarbocyanine perchlorate acetylated-low density lipoprotein (DiI-Ac-LDL; Biomedical Technologies, Stoughton, Mass., USA) and 10 μg/mL of FITC bound Ulex europaeus agglutinin 1 lectin (UEA, Sigma-Aldrich). Incorporation of DiI-Ac-LDL and bound FITC-UEA-1 was assessed using flow cytometry and compared to unlabelled control cells.

### 3.0 Results:

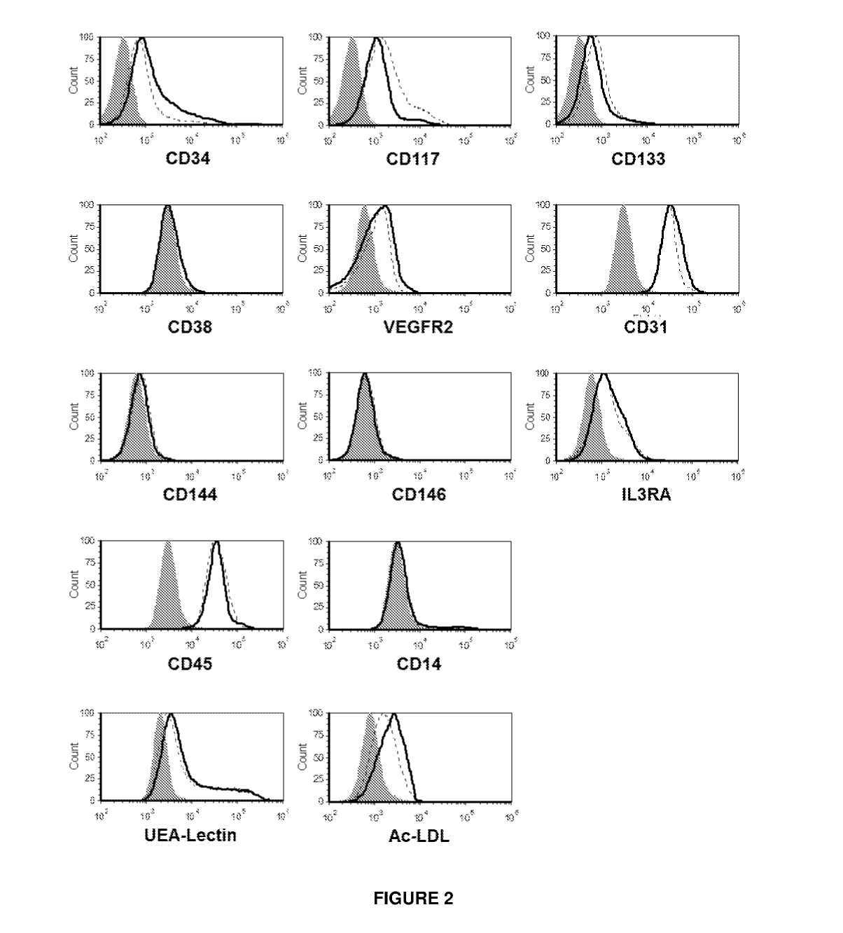

Between days 5 and 9 of expansion the surface markers on cord blood-derived CD133-isolated non-adherent EPCs (cultured as described in Experiment 2) were analysed using flow cytometry (FIG. 2). The progenitor cell markers CD34, CD117 and CD133 (Timmermans et al., 2009) were investigated and found on non-adherent EPCs, although the expression of CD133 and CD34 diminished over time as cells expanded, while CD117 remained constant, most likely as it is the receptor for SCF which is present within the CellGro media.

A number of cell surface mature endothelial vascular markers were investigated; VEGFR2, CD31, CD144 and CD146. VEGFR2 and CD31 were both present on expanded non-adherent EPCs, while CD144 and CD146 were absent (FIG. 2). VEGFR2 and CD31 are commonly found on various endothelial progenitors; while CD144 and CD146 are found on mature endothelial cells (Timmermans et al., 2009).

Monocyte and leukocyte markers were also examined. Expanded CD133+ non-adherent EPC showed a low level of CD45 expression, no CD38 expression and often contained a small population of CD14+ cells. Expanded non-adherent EPCs were positive for the uptake of Ac-LDL and binding UEA-1 lectin (FIG. 2), two features of EPCs and ECs (Timmermans et al., 2009).

Combined, these data suggest CD133-isolated cord blood cells expanded in CellGro produces a population of non-adherent EPCs, which express both hematopoietic progenitor and endothelial markers, while being deficient in monocyte markers. They express the classical EPC markers of CD117, VEGRF2 and CD31, while also having the ability to take up Ac-LDL and bind UEA-1 lectin.

Through flow cytometry we were able to confirm that expanded non-adherent EPCs express the alpha chain of the IL-3 receptor (IL-3RA, CD123). While IL-3 increased the rate of expansion, it was unable to cause any change in the cell surface marker expression of any progenitor, vascular or leukocyte markers. The IL-3RA expression, uptake of Ac-LDL and binding of UEA-1 lectin were all unchanged by the presence of IL-3 (FIG. 2).

### Example 4

### Immunogenicity of Non-Adherent EPCs

### 4.0 Methods:

### 4.1 Isolation and Culture of Human Umbilical Vein Endothelial Cells (HUVECs)

Primary HUVEC were extracted from human umbilical veins by collagenase digestion and cultured in HUVE media as previously described (Litwin et al., 1997; Wall et al., 1978) and were used no later than two passages.

### 4.2 Assessment of Immunogenicity of Non-Adherent EPCs

Non-adherent EPCs (isolated as described in Example 2) and HUVEC were isolated (as above) from the cord of donors and cultured for 1 week. As a positive control donor matched mature dendritic cells (DCs) were produced. Briefly, the CD133- fraction from non-adherent EPC separation was washed in PBS/2% FBS and centrifuged for 10 min at 120×g with no brake. Cells (5×10⁷) were suspended in 10 ml of RPMI Media 1640 (Life Technologies, Carlsbad, Calif.)/1% HI FBS and placed into a T75 flask for 45 min under cell culture conditions (37° C., 5% CO2). Non-adherent cells were removed with PBS washes and 10 ml of new media was added (RMPI+10% FBS, 400 U/ml of IL4 (eBioscience, San Diego, Calif.) and 800 U/ml of GM-CSF (R&D Systems). Cells were incubated for 5 days to produce immature DCs before adding 10 ng/ml of TNFα, IL6 (both R&D Systems), IL1β (PeproTech, Rocky Hill, N.J.) and PGE2 (Sigma-Aldrich) to generate mature DCs (mDCs) after a further 48 hr of incubation. Mature DCs (mDCs) were confirmed by flow cytometry, with cells being labelled with antibodies against HLA-DR, CD11c, CD14, CD80, CD83, CD86 (all BD Biosciences) and CD209 (Miltenyi Biotech).

Donor matched HUVEC (P1 or P2), non-adherent EPCs expanded for 7 days and mDCs and unmatched bone marrow-derived mesenchymal stem cells (MSC, Millipore) were labelled with mouse anti-human antibodies against MHC class I (clone FMC16, in house), MHC class II or the isotype control. Following incubation (30 min, 4° C.) cells were washed in media and labelled with goat anti-mouse Dylight650 secondary antibody (1/100 dilution in media, Abcam) for 30 min at 4° C. Cells were washed, labelled with 7AAD, washed again and then analyses by FACS.

In some experiments EPCs, HUVEC and MSC were cultured with 10 ng/ml of TNFα or IFNγ (R&D Systems) for 48 hrs prior to antibody labelling and FACS analysis.

### Results:

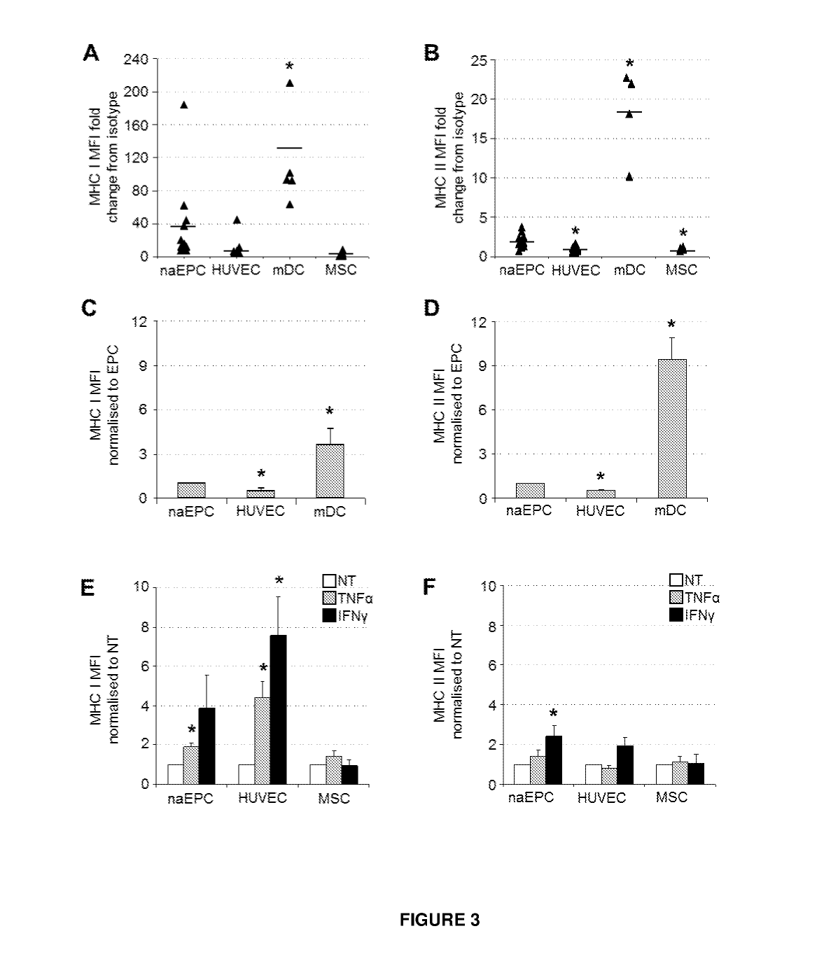

The immunogenicity of CD133-isolated and expanded non-adherent EPCs is of key importance for their use in any potential therapeutics. MHC class I and class II expression was assessed on non-adherent EPCs and compared to donor matched HUVEC and mDCs, and unmatched MSCs. The production of mDCs was confirmed by their high expression of HLA-DR, CD80, CD83 and CD86, lower expression of CD209 and CD11c compared to immature DCs and lack of CD14 expression. When data is expressed as a fold change in MFI compared to the isoptye control, non-adherent EPCs, HUVEC and MSCa show no difference in MHC class I expression, while the positive control, mDCs, have high MHC class I expression (FIG. 3A). However, when the MFI fold change of each HUVEC and mDC line is normalised to its donor-matched non-adherent EPCs, HUVEC express less and mDCs express more, MHC class I compared to expanded non-adherent EPCs (FIG. 3C).

For MHC class II, data expressed as a fold change in MFI compared to the isotype control (FIG. 3B) has non-adherent EPCs expressing more class II than both HUVEC and MSC, but significantly less than the positive control mDC. When the MFI fold change of HUVEC and mDCs is normalised to its donor-matched non-adherent EPCs, HUVEC express less, while mDCs express more, MHC class II compared to expanded non-adherent EPCs (FIG. 3D).

A site of vascular injury is an inflammatory environment, expressing a range of cytokines such as TNFα and IFNγ (Sprague et al., 2009). Therefore, it was of interest to assess the presence of MHC on EPCs, HUVEC and MSC, as a comparator cell, after the cells had been exposed to TFNα or IFNγ for 48 hr.

Each cell type cultured with cytokine was normalised to cells cultured without cytokine. MHC class I was significantly increased in non-adherent EPCs exposed to TFNα and there was a trend towards an increase in response to IFNγ. However, the effect was not as profound as that seen in HUVEC. HUVEC significantly increased MHC class I in response to TNFα and IFNγ. In addition, the extent of the increase tended to be less in non-adherent EPCs compared to HUVEC (MHC I: 1.9±2.1 vs 4.4±0.9; MHC II: 3.9±1.6 vs 7.6±2.0), indicating that HUVEC may be more responsive to TNFa and IFNy than non-adherent EPCs. The expression of MHC class I was unaffected on MSCs by cytokine (FIG. 3E).

MHC class II was only increased in non-adherent EPCs cultured with IFNγ, TNFα had no effect. Neither cytokine changed the expression profile of MHC II on HUVEC or MSC. The induction of MHC II was less than the MHC I upregulation (FIG. 3F).

### Example 5

### Vascular Properties of Expanded Non-Adherent EPCs

### 5.0 Methods:

### 5.1 In Vitro Matrigel™ Tube Formation Assay

In vivo Matrigel™ plug assays utilized 6-8 week old female NOD/SCID mice. Rodents were purchased from the Animal Resources Centre (Perth, Australia) and were housed in specific pathogen-free conditions in the SA Pathology Animal Care Facility (Adelaide, Australia). In vitro tube formation of HUVEC and expanded non-adherent EPCs was assessed. HUVEC were stained with 10 μg/ml DiI-Ac-LDL for 4 hours under cell culture conditions, washed once and incubated overnight under cell culture conditions. Non-adherent EPCs (cultured as described in example 2) were stained with 10 μM Calcein-AM (eBioscience, San Diego, Calif.) for 20 min at room temperature. Matrigel™ (10 μl, BD Biosciences), an extracellular matrix derived from murine sarcoma cells that supports vascular tube formation, was pippetted into the wells of an angiogenesis (ibidi GmbH, Martinsried, Germany). Matrigel™ was allowed to set for 30 min at 37° C. Labelled HUVEC and naEFCs were seeded alone or together onto the set Matrigel™ at a variety of cells densities. Tube formation was monitored regularly and 7 overlapping phase contrast images were captured using an inverted IX70 microscope 4×/0.13NA objective, an S15 F view camera and Analysis Life Sciences software (Olympus, Tokyo Japan) after 6 hours of tube formation. These overlapping images were “stitched” together using Adobe Photoshop (Adobe Systems, San Jose, Calif.) and the number of tubes, branches and loops were quantified manually using ImageJ 1.47 (National Institute of Health, Bethesda, Md.). Fluorescent images were captured on IX71 microscope (Olympus) with 10×/0.4NA objective and a Hamamatsu Orca-ER camera using the Analysis Life Sciences software.

### 5.2 In Vivo Matrigel™ Plug Tube Formation Assay

To assess the contribution of expanded non-adherent EPCs to tube-like structure formation in vivo, CD133⁺ non-adherent EPCs were expanded for 6-8 days before 5×10⁵ non-adherent EPCs were mixed in 500 μl of Matrigel™ with 2 μg/ml basic fibroblast growth factor (R&D Systems) and 50 units/ml heparin (Sigma-Aldrich) and injected subcutaneously into one flank of a female NOD/SCID mouse. The opposite flank received a control Matrigel™ plug containing no cells. Mice were humanely killed by cervical dislocation either on day 7, 13 or 19 post Matrigel™ injection. Plugs were removed, washed in PBS, fixed in 4% paraformaldehyde (VWR International, Radnor, Pa.), embedded in paraffin, cut into 8 μM sections on a microtome (RM 2235 Leica, Solms, Germany) and placed onto slides (Polysine®, Menzel-Glaser, Thermo Scientific).

Sections were microwaved in a citrate buffer for antigen retrieval and quenched of endogenous preoxidase activity with a 30 min incubation in H₂O₂. Sections were blocked with rabbit serum (Sigma-Aldrich) for 60 min prior to an overnight incubation in a humidity chamber with goat anti-mouse/human CD31 (Santa Cruz, Dallas, Tex.) diluted 1:1000 with PBS+3% normal rabbit serum. Sections were washed and incubated for 35 min with biotinylated rabbit anti-goat (Abcam) diluted 1:500 with PBS+3% normal human serum (Life Technologies, Carlsbad, Calif.). Sections were treated with Vectastain elite ABC regent (Vector Labs, Burlingame, Calif.) for DAB staining as per manufacturer's instructions, followed by haematoxlin and eosin staining. Negative controls were secondary antibody alone.

Some sections were stained with a mouse monoclonal antibody (MTC02, Abcam), which recognises a 60 kDa non-glycosylated protein component of mitochondria found only in human cells, in order to distinguish mouse and human cells within the plug. Briefly, paraffin sections were dewaxed in xlyene and endogenous peroxidise blocked with 0.5% H₂O₂. MTC02 antibody was diluted 1/500 in PBS+3% normal mouse serum and incubated o/n. Sections were incubate with a biotinylated goat anti-mouse secondary (Vector Labs) used at 1/250 in PBS/3% NHS. Sections were rinsed with PBS treated with ABC reagent as above.

Images of slides were collected using a Hamamatsu Nanozoomer slide scanner with CD31+ vessels counted manually and section areas quantified using the NDP view software (Hamamatsu Photonics, Hamamatsu, Japan).

### 5.0 Results:

A key feature of mature ECs and some progenitor cells, such as ECFCs, is their ability to form tube-like structures on Matrigel™ in vitro. HUVEC (1×10⁴ cells) were co-cultured with 1×10⁴ non-adherent EPCs that had been expanded in the presence or absence of IL-3. Control wells contained HUVEC alone either at 1×10⁴ or 2×10⁴ cells/well. Each donor HUVEC line was performed in triplicate, with tube structures, tube branching points (branches) and loops (a completely enclosed circuit made from tubes) counted manually at 6 hr and normalised to 2×10⁴ HUVEC.

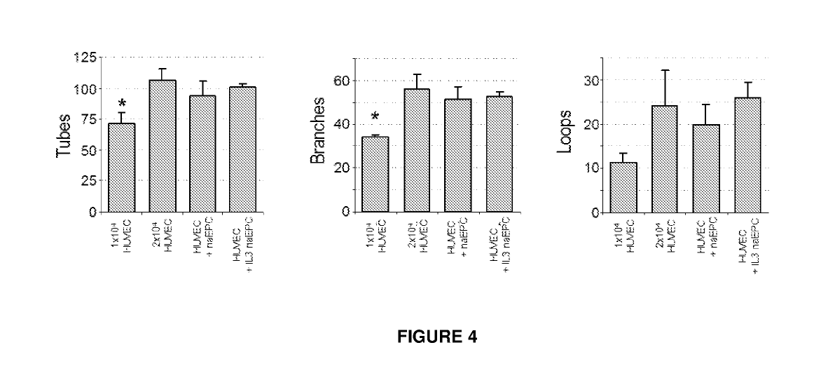

As expected, wells containing 1×10⁴ HUVEC formed fewer tubes, loops and branches than wells containing 2×10⁴ HUVEC. Wells containing the co-culture of HUVEC and non-adherent EPCs formed the same number of tubes, branches and loops as the control wells containing 2×10⁴ HUVEC (FIG. 4A). Wells containing 1×10⁴ HUVEC and 1×10⁴ non-adherent EPCs had the same number of cells as 2×10⁴HUVEC wells, but contained half as many HUVEC, however the co-culture produced the same number of tubes, branches and loops. This suggests that the non-adherent EPCs were able to contribute to the tube structure formation. There was no difference between non-adherent EPCs expanded in IL-3 deficient or replete CellGro media in their ability to contribute to tube, branch or loop formation (FIG. 4A).

In addition to the data suggesting the non-adherent EPCs assist in HUVEC tube formation, calcein-AM labelled non-adherent EPCs are closely associated with Ac-LDL labelled HUVEC further indicating that non-adherent EPCs can assist in tube formation. The non-adherent EPCs are not randomly scattered around the well, but are in close proximity to the HUVEC tubes. Expanded non-adherent EPCs alone on Matrigel™ retain their round appearance and do not form any tube-like structures, irrespective of IL-3 exposure.

Previously we have shown that a different population CD133-isolated cord blood cells that were not expanded and non-adherent EPCs do not form tube structures by themselves in vitro, but do form vessels with a lumen in vivo (Appleby et al., 2012). Non-adherent EPCs expanded according to methods described herein do not form tube structures in vitro. The ability of the expanded non-adherent EPCs to contribute to vessel formation in vivo was tested using Matrigel™ plugs containing expanded non-adherent EPCs or control plugs containing only PBS which were injected into each flank of female NOD/SCID mice. Plugs were removed, paraffin embedded, sectioned and staining with anti-mouse/human CD31 or MTC02, a human mitochondrial specific marker.

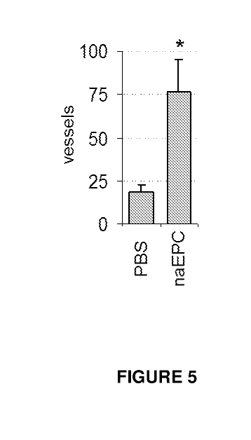

While, in these xenogeneic experiments, the human non-adherent EPCs do not directly from vessels they do contribute to vessel formation. This is illustrated by non-adherent EPC containing plugs having more vessels in the tissue surrounding the plug compared to the tissue surrounding non-adherent EPC-deficient plugs (FIG. 5).

### Example 6

### Administration of Expanded Non-Adherent EPCs to Rats with Acute Myocardial Infarction

### 6.1 Methods:

### 6.0 Acute Myocardial Infarction in CBH-Rnu Rats

Myocardial infarction experiments utilized 8-10 week old male CBH-Rnu rats. Rodents were purchased from the Animal Resources Centre (Perth, Australia) and were housed in specific pathogen-free conditions in the SA Pathology Animal Care Facility (Adelaide, Australia). CBH-Rnu male rats were anaesthetised using isoflurane (1% in 3-4 L O₂/min, Bomac Laboratories, Sydney, Australia) via nose cone. Once ventilated, rats underwent a permanent surgical ligation of the left anterior descending coronary artery to induce acute myocardial infarction (AMI). Rats then received either PBS or 1×10⁶ non-adherent EPCs in 100 μl by transepicardial injection directly below the ligation site before the wound was closed in 3 layers. Rats were allowed to recover on 100% oxygen and received Carprofen (5 mg/kg) and Norocillin (10 mg/kg, both Norbrook Laboratories, Melbourne, Australia). Following a cardiac magnetic resonance image (MRI) at 7 days post AMI, blood was collected from tail veins for serum creatinine detection by a colorimetric test at IMVS Pathology (Adelaide, Australia) before humane killing. Hearts were collected for histology and RNA isolation.

### 6.1 Cardiac Magnetic Resonance Imaging

Magnetic resonance imaging was performed on a 1.5T MR system (Magnetom Sonata, Siemens, Germany). Cardiac MRI was performed on rats immediately prior to AMI and one week post-AMI. Animals were placed supine recumbent in the isocentre of the magnet with a human carotid radiofrequency coil placed over the thorax. Anaesthesia was maintained by isoflurane. Two electrocardiography (ECG) electrodes were attached to the thorax thereby generating a vector ECG. Accordingly, all MRI images were free breathing, ECG-gated, acquisitions. Transverse and coronal localiser images were acquired followed by short axis pilot images, from which a true short axis stack was prescribed. TrueFISP (Fast Imaging with Steady-State Precession) cine images (gated to alternate R waves) were acquired. The stack comprised three contiguous left ventricular slices (each 3 mm thick, with no intersection gap) providing almost complete coverage of the left ventricle. The image matrix was 384×384, field of view 185 mm, repetition time 14.72 ms, echo time 1.55 ms, flip angle 90°, and 20 heart phases were acquired. The average scan time per animal was 10 min.

### 3.2 MRI Image Analysis

Left ventricular volumes and derived ejection fraction (EF) were measured off-line from cine images using commercially available software (QMASS v7.2, Medis, Netherlands). Papillary muscles were excluded from calculations. The end-diastolic (ED) and end-systolic (ES) cine frames were identified for each slice and the endocardial and epicardial borders were manually traced. The end-diastolic (EDV) and end-systolic volumes (ESV) were then calculated using the true disk summation technique (sum of cavity volumes across all continuous slices), as previously described (Teo et al., 2008).

6.3 PCR of Heart Tissue Collected from Acute Myocardial Infarction Rats

Quantification of mRNA levels was carried out using qPCR. Primers designed for rat genes using Primer Blast (National Institute of Health) and purchased from GeneWorks (Adelaide, Australia). Where possible, primers were designed to span an intron/exon border to ensure no genomic DNA amplification. qPCR amplification was performed using QuantiTect™ SYBR Green master mix (Qiagen, Venlo, Netherlands) on a Rotor-Gene thermocycler (Corbett Research, Mortlake, Australia) with reaction parameters: 15 minutes at 95° C., then cycling of 10 seconds 95° C., 20 seconds 55° C. and 30 seconds 72° C.; for 45 cycles followed by a melt phase. Data obtained was analysed using Rotor-Gene Analysis Software version 6 (Corbett Research). Relative gene expression levels were calculated by normalising to the rat house-keeping genes β-actin, CycA, HPRT and YWAHZ using the geNorm software.

TABLE 1
Rat primers used in PCR analysis of gene
expression in AMI cardiac tissue

| Rat Gene | Forward and Reverse Prime Sequence |
|---|---|
| TNFα | F-5′AAAGCATGATCCGAGATGTG3′ |
| | R-5′AGCAGGAATGAGAAGAGGCT3′ |
| HSP70 | F-5′GTGAACTACAAGGGCGAGAA3′ |
| | R-5′CGATCTCCTTCATCTTGGTCAG3′ |
| SDF1 | F-5′CCGATTCTTTGAGAGCCATGT3′ |
| | R-5′CAATGCACACTTGTCTGTTGTT3′ |
| VEGFR2 | F-5′GACGACCCATTGAGTCCAATTA3′ |
| | R-5′GTGAGGATGACCGTGTAGTTTC3′ |
| CX3CL1 | F-5′GCAGCTTTCCGCTCTGAATA3′ |
| | R-5′CTGTTCTGAGCTTCCACCTATC3′ |
| CD14 | F-5′GGAAAGAAACTGAAGCCTTTCTC3′ |
| | R-5′AGCAACAAGCCGAGCATAA3′ |
| IL6 | F-5′AAGAGACTTCCAGCCAGTTGCC3′ |
| | R-5′ACTGGTCTGTTGTGGGTGGTATC3′ |
| IL8 | F-5′GCACCCAAACCGAAGTCATA3′, |
| | R-5′GGGACACCCTTTAGCATCTTT3′ |
| CCL2 | F-5′GTCTCAGCCAGATGCAGTTAAT3′ |
| | R-5′CTGCTGGTGATTCTCTTGTAGTT3′ |
| MMP3 | F-5′GATGATGAACGATGGACAGATGA3′ |
| | R-5′AGCATTGGCTGAGTGAAAGA3′ |
| MMP9 | F-5′CAACCTCACGGACACACA3′, |
| | R-5′CTGCTTCTCTCCCATCATCTG3′ |
| β-actin | F-5′CTTCCTTCCTGGGTATGGAATC3′, |
| | R-5′CTGTGTTGGCATAGAGGTCTT3′ |
| CycA | F-5′GGCTATAAGGGTTCCTCCTTTC3′ |
| | R-5′TTGCCACCAGTGCCATTA3′ |
| HPRT | F-5′GACCTCTCGAAGTGTTGGATAC3′ |
| | R-5′TCAAATCCCTGAAGTGCTCAT3′ |
| YWAHZ | F-5′GACATCTGCAACGACGTACT3′, |
| | R-5′CGGTAGTAGTCACCCTTCATTT3′ |

### 6. 4 Statistical Analysis

Results are generally expressed as mean±standard error of the mean (SEM). An unpaired Student T-test, 1- or 2-way ANOVA for multiple comparisons was performed to determine statistical significance between groups with p values<0.05 considered significant.

### 6.0 Results:

CBH-Rnu male rats underwent a surgical ligation of the left anterior descending coronary artery to induce myocardial infarction. Rats then received either PBS or non-adherent EPCs injected transepicardial below the ligation site. Seven days pre- and post-surgery rats were analysed by MRI to measure left ventricular mass (LVM) and ejection fraction (EF). Blood was also collected to assess serum creatinine.

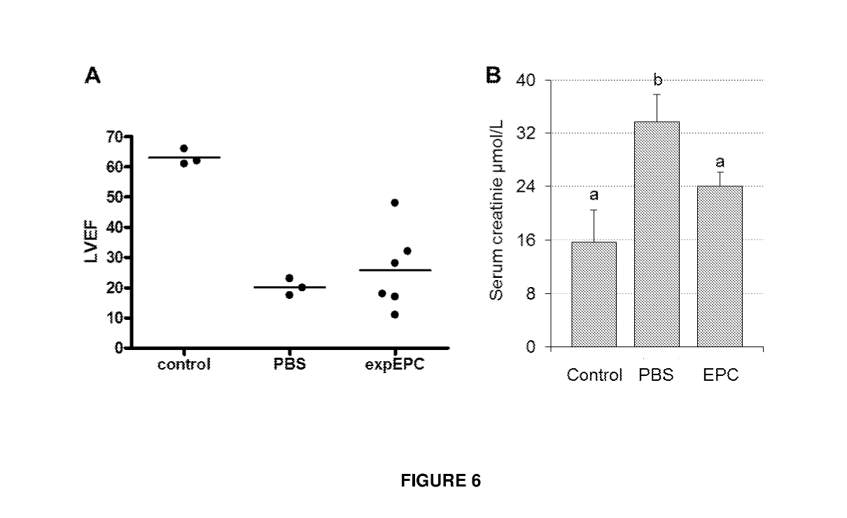

Irrespective of whether rats received PBS or non-adherent EPCs, surgery reduced the EF in rats (FIG. 6A) as expected. In comparison to PBS, administration of non-adherent EPCs produced a clinically beneficial increase in LVEF. Delivery of non-adherent EPCs did not improve LVM.

Creatine phosphate in muscle is broken down into creatinine that enters the blood stream. While this is a constant process within the body, high levels of serum creatinine are linked to cardiovascular distress. Surgery increased creatinine in rats treated with PBS compared to controls. The delivery of non-adherent EPCs returned creatinine to pre-AMI levels, suggesting that non-adherent EPCs were able to reduce the amount of cardiac damage induced by the AMI surgery (FIG. 6B).

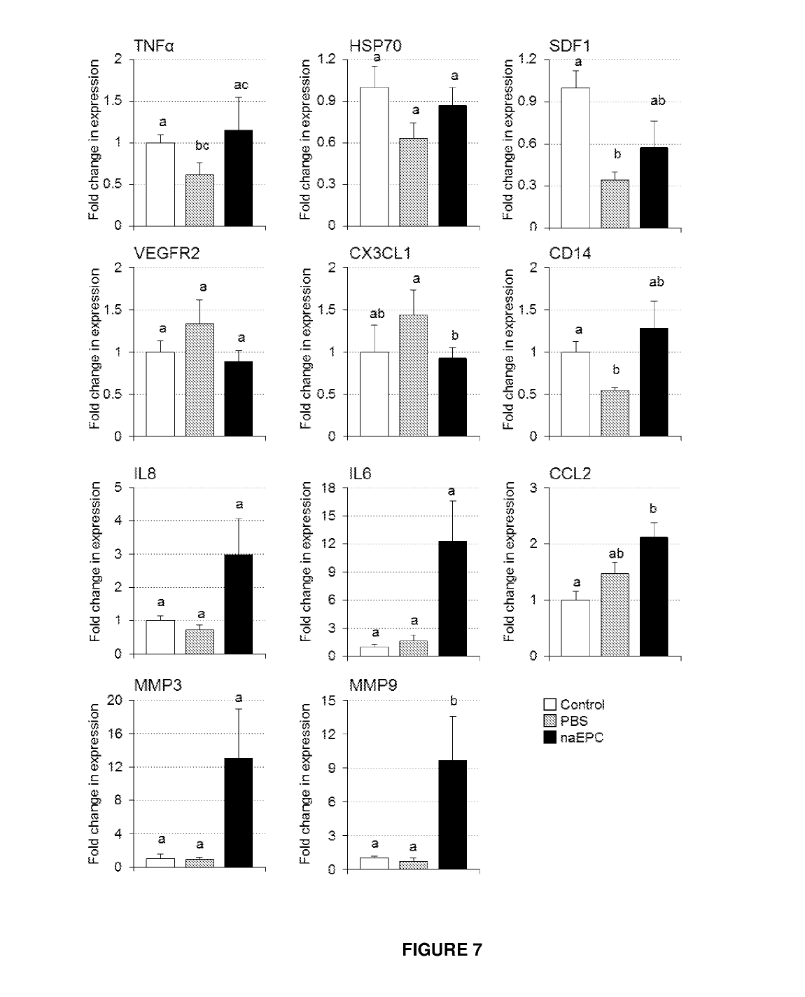

Cardiac tissue was collected and qPCR was utilized to measure endogenous rat mRNA of TNFA, HSP70, SDF1, VEGFR2, CX3CL1, CD14, IL6, IL8, CCL2, MMP3 and MMP9. Expression was normalised to housekeeper genes and control rats not undergoing surgery (FIG. 7). Rats receiving non-adherent EPC treatment post-AMI had a number of genes that had a tendency to return to the control levels, namely TNFA, HSP70, VEGFR2, CX3CL1 and CD14. More significantly there were profound (even if not always statistically significant due to low numbers) increases in a number of genes with both proinflammatory and proangiogenic properties, namely IL8, IL6, CCL2, MMP3 and MMP9. In vitro and in vivo the non-adherent EPCs have displayed proangiogenic properties and the mRNA analysis reveals that they can induce expression of genes that may play a role in AMI recovery

In conclusion, the methods provided in Examples 1 to 7 provide for the generation of large numbers of non-adherent EPCs that display proangiogenic properties in vitro and in vivo an suggest improved outcomes of rats with experimental surgically induced acute myocardial infarction.

### REFERENCES

Appleby et al, PLoS One.;7(11):e46996, 2012;
Asahara et al., Science, 275: 964-967, 1997;
Ausubel F. M. et al., (editors), Current Protocols in Molecular Biology, Greene Pub. Associates and Wiley-Interscience (1988, including all updates until present);
Autiero, Nat Med. July; 9(7):936-43, 2003;
Bagley et al., Cancer Res., 63: 5866, 2003;
Brown (editor), Essential Molecular Biology: A Practical Approach, Volumes 1 and 2, IRL Press (1991);
Carmeliet et al., J. Pathol., 190: 387-405, 2000;
Cima, et al., Biotechnol. Bioeng., 38:145 1991;
Coligan J. E. et al., (editors) Current Protocols in Immunology, John Wiley & Sons (including all updates until present);
Couffinhal et al., Am. J. Pathol., 152: 1667-1679, 1998;
Glover and Hames (editors), DNA Cloning: A Practical Approach, Volumes 1-4, IRL Press (1995 and 1996);
Harlow and Lane (editors) Antibodies: A Laboratory Manual, Cold Spring Harbour Laboratory, (1988);
Hill et al N Engl J Med., 2003; 348:593-600;
Kawamoto and Losordo, Trends Cardiovasc. Med., 18: 33-37, 2008;
Krenning et al., Trends in Mol. Med., 15: 180-189, 2009;
Li et al., J Immunol; 170 (6): 3369-3376, 2003;
Li et al., FASEB J, 20 (9): 1495-1497, 2006;
Litwin et al., J. Cell Biol. 139, 219-228, 1997;
Liu et al., Antioxid. Redox Signal., 10: 1869-1882, 2008;
Lyden et al., Nature Med., 7: 1194-1201, 2001;
Miltenyi et al., Cytometry, 11: 231-238, 1990;
Perbal (editor), “A Practical Guide to Molecular Cloning”, John Wiley and Sons (1984);
Sambrook et al. (editors), “Molecular Cloning: A Laboratory Manual”, Cold Spring Harbour Laboratory Press (1989);
Sprague et al., Biochem Pharmacol., 78(6):539-52, 2009;
Teo et al., Heart Lung Circ, 17 (4): 313-317, 2008;
Timmermans et al., J Cell Mol Med., 13(1):87-102, 2009;
Vacanti et al., J. Ped. Surg., 23: 3-9 1988;
Vacanti et al., Plast. Reconstr. Surg., 88: 753-9 1991;
Wall et al. J Cell Physiol., 96 (2): 203-213, 1978.

1. A method for producing a population of cells, enriched for non-adherent endothelial progenitor cells (EPCs), the method comprising culturing an EPC containing population of cells in the presence of interleukin-3 (IL-3), such that a population of cells enriched for non-adherent EPCs is produced.

2. The method of claim 1, additionally comprising culturing the EPC containing population of cells in the presence of one or more factors selected from the group consisting of thrombopoietin (TPO), Flt3-ligand (Flt3L) and stem cell factor (SCF).

3. The method of claim 1, additionally comprising culturing the EPC containing population of cells in the presence of placental growth factor (PIGF).

4. The method of claim 1, additionally comprising culturing the EPC containing population of cells in a medium comprising IL-3.

5. The method of claim 4, wherein the medium additionally comprises, TPO, Flt3L and SCF.

6. The method of claim 5, wherein the medium additionally comprises PIGF.

7. The method of claim 4, wherein the medium is serum-free.

8. The method of claim 1, additionally comprising culturing the EPC containing population of cells in a serum-free medium comprising IL-3, TPO, Flt3L and SCF.

9. The method of claim 8, wherein the serum-free medium additionally comprises PIGF.

10. The method of claim 1, wherein the EPC containing population of cells are cultured under conditions such that they remain non-adherent.

11. The method of claim 1, further comprising adding additional IL-3 to the EPC containing population of cells, wherein the additional IL-3 is added following expansion of the EPC population.

12. The method of claim 11, wherein the additional IL-3 is added without removing previous IL-3 from the EPC containing population of cells.

13. The method of claim 1, additionally comprising isolating and/or expanding the population of cells enriched for non-adherent EPCs.

14. The method of claim 13, wherein the population of cells enriched for non-adherent EPCs are isolated by isolating DSG2+EPCs.

15. The method of claim 13, wherein the population of cells enriched for non-adherent EPCs are isolated by isolating CD133+EPCs.

16. The method of claim 1, additionally comprising formulating the population of cells enriched for non-adherent EPCs into a pharmaceutical formulation.

17. A method of producing a pharmaceutical formulation, the method comprising:
obtaining a population of cells enriched for non-adherent EPCs produced by the method of claim 1; and
formulating the population of cells enriched for non-adherent EPCs into a pharmaceutical formulation.

18. A method of treating a subject, the method comprising performing the method of claim 17 and administering the population of cells enriched for non-adherent EPCs to the subject.

19. A method of treating a subject, the method comprising obtaining a pharmaceutical formulation produced by the method of claim 17 and administering the pharmaceutical formulation to the subject.
